(19) United States
(12) Patent Application Publication
(10) Pub. No.: US 2026/0051370 A1
(43) Pub. Date: Feb. 19, 2026

(54) GENERATIVE DESIGN OF SMALL MOLECULES

(71) Applicant: Genentech, Inc., South San Francisco, CA (US)

(72) Inventors: Paul Powell BEROZA, Belmont, CA (US); Lev Yuryevich GENDELEV, Vallejo, CA (US)

(73) Assignee: Genentech, Inc., South San Francisco, CA (US)

(21) Appl. No.: 19/320,786

(22) Filed: Sep. 5, 2025

Related U.S. Application Data
(63) Continuation of application No. PCT/US2024/019256, filed on Mar. 8, 2024.
(60) Provisional application No. 63/489,648, filed on Mar. 10, 2023.

(51) Int. Cl. G16C 20/30; G16C 20/50; G16C 20/70

(52) U.S. Cl. CPC G16C 20/30; G16C 20/50; G16C 20/70

(57) ABSTRACT

Methods for the generative design of small molecules include performing, until one or more conditions are satisfied, one or more iterations of a generative algorithm. Each iteration of the generative algorithm may include modifying one or more molecules from an initial population of molecules. Moreover, each iteration of the generative algorithm may include selecting, from the initial population of molecules and the one or more modified molecules, a quantity of molecules satisfying one or more fitness scores for inclusion in a subsequent population of molecules. If the one or more conditions are not satisfied, one or more additional iterations of the generative algorithm may be performed using a different initial population of molecules or the subsequent generation of molecules as a new initial population of molecules. Related systems and computer program products are also provided.

### TECHNICAL FIELD

The subject matter described herein relates generally to small molecule design and more specifically to iterative methods for generating a set of molecules that bind to a particular target.

### BACKGROUND

Small molecule drugs, which typically have a molecular weight between approximately 100 Daltons and 1,000 Daltons, modulate biochemical processes to diagnose, treat, and prevent a gamut of illnesses. Such molecules have been a cornerstone of modern pharmacology due to a number of compelling advantages, such as significant flexibility in tailoring their formulation and pharmacokinetics. For example, small molecule drugs are capable of penetrating cell membranes to reach intracellular targets. Moreover, small molecule drugs are adaptable to a wide variety of therapeutic applications. For instance, a small molecule drug may be formulated as pills and capsules, intravenous or subcutaneous injectables, inhalational medicines, or suppositories. Consequently small molecule drugs have been designed to suit a wide variety of therapeutic applications. The development of a small molecule drug typically includes achieving a particular specificity of binding to a particular target, in conjunction with optimizing various pharmacokinetic properties including liberation, absorption, distribution, metabolism, and excretion.

However, developing a small molecule drug to exhibit certain desirable properties, such as an ability to bind to a target site, is a challenging and resource intensive task at least because the chemical space of all possible drug-like molecules is vast but only very sparsely populated by potential drug candidates with the necessary binding affinity to any given target. Given that the overwhelming majority of the estimated 10³³ to 10⁶⁰ drug-like molecules in chemical space will have no medicinal value, identifying the few molecules in that space that are potential drug candidates typically requires exploring as much of the chemical space as efficiently as possible.

Nevertheless, a brute force approach to indiscriminately screen every drug-like molecule to identify candidate molecules capable of binding to the target site, even if it were to be performed in silico, is too computationally expensive to be a practical solution.

### SUMMARY OF THE DISCLOSURE

Systems, methods, and articles of manufacture, including computer program products, are provided for the generative design of small molecules. In one aspect, there is provided a system that includes at least one processor and at least one memory. The at least one memory may includes program code that provides operations when executed by the at least one processor.

In another aspect, there is provided a computer program product including a non-transitory computer readable medium storing instructions. The instructions may cause operations to be executed by at least one data processor.

In another aspect, there is provided a system for generative design of small molecules that includes at least one processor and at least one memory. The at least one memory may include program code that provides operations when executed by the at least one processor.

In another aspect, there is provided a computer-implemented method, comprising:
(a) obtaining, from a molecular structure database, an initial population of molecules;
(b) generating a first new population of molecules by modifying at least a first molecule in the initial population of molecules and selecting, from the initial population of molecules and at least the first modified molecule, a quantity of molecules satisfying one or more fitness scores;
(c) generating a second new population of molecules by modifying at least a second molecule in the first new population of molecules and selecting, from the first new population of molecules and at least the second modified molecule, another quantity of molecules satisfying the one or more fitness scores;
(d) in response to determining that a convergence criterion has not been satisfied, repeating step (c) at least once with the second new population of molecules as a first new population of molecules; and
(e) in response to determining that the convergence criterion has been satisfied, selecting a subset of the second new population of molecules as candidates for synthesis and testing.

In another aspect, there is provided a computer-implemented method, comprising:
(a) obtaining, from a molecular structure database, a plurality of initial populations of molecules;
(b) generating a plurality of first new populations of molecules from each of the plurality of respective initial populations of molecules, wherein each of the plurality of first new populations of molecules comprises one or more molecules obtained by modifying one or more molecules in the initial population of molecules on which the first new population is based, wherein the molecules in each of the plurality of first new populations of molecules satisfy one or more fitness scores;
(c) generating a plurality of second new populations of molecules from each of the respective plurality of first new populations of molecules, wherein each of the plurality of second new populations comprise one or more molecules obtained by modifying one or more molecules in the first new population of molecules on which the second new population is based, wherein the molecules in each of the plurality of second new populations of molecules satisfy the one or more fitness scores;
(d) in response to determining that a convergence criterion has not been satisfied, repeating step (c) at least once with each of the first new populations of molecules being one of the second new populations of molecules obtained from the prior instance of step (c); and
(e) in response to determining that the convergence criterion has been satisfied, selecting a subset of molecules that comprises at least one molecule from each of the plurality of second new populations of molecules for synthesis and testing.

In another aspect, there is provided a computer-implemented method, comprising:
(a) obtaining an initial population of molecules wherein each molecule has a molecular structure constructed from a reaction database that contains a set of reactions, wherein each reaction from the set of reactions is associated with two or more sets of reagents, and wherein each molecule in the initial population of molecules is a product of a first reagent from a first set of reagents and a second reagent from a second set of reagents, and optionally a third reagent from a third set of reagents, in accordance with a reaction to which both the first and second reagents and the optional third set of reagents are associated;
(b) generating a first new population of molecules by
   modifying at least a first molecule in the initial population of molecules by replacing the first reagent from which the first molecule is formed with another first reagent from the first set of reagents associated with the reaction from which the first molecule is formed, and optionally replacing the second reagent from which the first molecule is formed with another second reagent from the second set of reagents associated with the reaction from which the first molecule is formed, thereby forming a first modified molecule, and
   selecting, from the initial population of molecules and at least the first modified molecule, a quantity of molecules that satisfy one or more fitness scores for inclusion in the first new population of molecules;
(c) generating a second new population of molecules by at least
   modifying at least a second molecule in the first new population of molecules by replacing the first reagent from which the second molecule is formed with another first reagent from the first set of reagents, and optionally replacing the second reagent from which the second molecule is formed with another second reagent from the second set of reagents thereby forming a second modified molecule, and
   selecting, from the first new population of molecules and at least the second modified molecule, another quantity of molecules that satisfy the one or more fitness scores;
(d) in response to determining that a convergence criterion has not been satisfied, repeating step (b) at least once with the second new population of molecules as a new initial population of molecules; and
(e) in response to determining that the convergence criterion has been satisfied, selecting a subset of the second new population of molecules as candidates for synthesis and testing.

In another aspect, there is provided a computer-implemented method, comprising:
(a) obtaining, from a molecular structure database, an initial population of molecules, each molecule of the initial population of molecules being a product of a first reaction between a first reagent selected from a first set of reagents associated with the first reaction and a second reagent selected from a second reagent selected from a second set of reagents associated with the first reaction, and optionally a third reagent from a third set of reagents associated with the first reaction;
(b) generating a first new population of molecules by
   creating a first modified molecule by applying a modification to at least a first molecule in the initial population of molecules, wherein the modification is selected from one or more of:
      replacing the first reagent from which the first molecule is formed with another first reagent selected from the first set of reagents;
      replacing the second reagent from which the first molecule is formed with another second reagent selected from the second set of reagents; and
      replacing a third reagent from which the first molecule is formed with another third reagent selected from the third set of reagents;
   and
   selecting, from the initial population of molecules and at least the first modified molecule, a quantity of molecules satisfying one or more fitness scores;
(c) generating a second new population of molecules by
   creating a second modified molecule by applying a modification to at least a second molecule in the second new population of molecules, wherein the modification is selected from one or more of:
      replacing the first reagent from which the first molecule is formed with another first reagent selected from the first set of reagents;
      replacing the second reagent from which the first molecule is formed with another second reagent selected from the second set of reagents; and
      replacing a third reagent from which the first molecule is formed with another third reagent selected from the third set of reagents;
   and
   selecting, from the first new population of molecules and at least the second modified molecule, another N quantity of molecules satisfying the one or more fitness scores;
(d) in response to determining that a convergence criterion has not been satisfied, repeating step (c) at least once with the second new population of molecules as a new initial population of molecules; and
(e) in response to determining that the convergence criterion has been satisfied, selecting a subset of the second new population of molecules as candidates for synthesis and testing.

In another aspect, the one or more fitness scores include one or more diversity scores.

In another aspect, there is provided a system, comprising at least one data processor; and at least one memory storing instructions, which when executed by the at least one data processor, result in operations comprising any of the methods described herein.

In another aspect, there is provided a non-transitory computer readable medium storing instructions, which when executed by at least one data processor, result in operations comprising any of the methods described herein.

Implementations of the current subject matter can include, but are not limited to, systems and methods consistent with including one or more features described herein, as well as articles that comprise a tangibly embodied machine-readable medium operable to cause one or more machines (e.g., computing devices as elsewhere described herein) to result in operations described herein. Similarly, computer systems are also described that may include one or more processors and one or more memories coupled to the one or more processors. A memory, which can include a computer-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the design of small molecules, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a small molecule design system, in accordance with some example embodiments;

FIG. 2 illustrates examples of two sets of reagents and a reaction between them to form a product, depicted both chemically and computationally, in accordance with some example embodiments;

FIGS. 3A and 3B illustrates schematics for two variants of a method of modifying molecules that are constructed from reagents according to a reaction, in accordance with some example embodiments;

FIG. 4 depicts a single iteration of an example of a generative algorithm for small molecule design, in accordance with some example embodiments;

FIG. 5 depicts multiple iterations of an example of a generative algorithm for small molecule design in which one or more subsequent populations of molecules are generated based on a single initial population of molecules, in accordance with some example embodiments;

FIG. 6 depicts multiple iterations of an example of a generative algorithm for small molecule design in which one or more subsequent populations of molecules are generated based on multiple initial populations of molecules, in accordance with some example embodiments;

FIG. 7 depicts graphs illustrating the evolution of molecule properties across successive populations of molecules, in accordance with some example embodiments;

FIG. 8A depicts an example of an application of a generative small molecule design method as described herein in which a generated molecule is evaluated for consistency to one or more known molecules, in accordance with some example embodiments;

FIG. 8B depicts another example of an application of a generative small molecule design method as described herein in which a generated molecule is evaluated for novelty, in accordance with some example embodiments;

FIG. 9A depicts a flowchart illustrating an example of a process for designing small molecules, in accordance with some example embodiments;

FIG. 9B depicts a flowchart illustrating another aspect of an examplary process for designing small molecules, in accordance with some example embodiments; and

FIG. 10 depicts a block diagram illustrating an example of a computing system, in accordance with some example embodiments.

When practical, similar reference numbers denote similar structures, features, or elements.

### DETAILED DESCRIPTION OF THE DISCLOSURE

The subject matter herein relates to iterative methods for generating a set of molecules that have been optimized to bind to (e.g., active against) a particular target and/or to have properties that are likely to make them promising drug candidates. The methods described herein address, for example, the problem identifying a set of molecules including two or more molecules that are promising drug candidates while also being diverse and readily available to be synthesized (e.g., economical to manufacture), as compared to known methods generating molecules that lack at least one of these characteristics, for example, lack molecular diversity and/or are so structurally complex that it is chemically impractical to synthesize them.

In some embodiments, the methods described herein address this problem by iteratively applying a generative algorithm to two or more populations of molecules to generate molecules of subsequent populations of the two more populations, wherein the generating the molecules of the subsequent populations includes a selection based on a diversity score to, for example, maximize inter-population diversity. In some embodiments, the generating the molecules of the subsequent populations includes a selection based on another diversity score to, for example, maximize intra-population diversity. In some embodiments, the diversity score includes a fitness score. The terms “diverse” or “dissimilar” are used herein interchangeably. In some embodiments, the diverse molecules are structurally and/or chemically dissimilar.

In some embodiments, the generating the molecules of the subsequent populations includes a selection based on a fitness score to, for example, optimize binding of the generated molecules to a particular target and/or having properties that are likely to make the generated molecules promising drug candidates. In some embodiments, the fitness score differs from the diversity score used to, for example, maximize inter-population and/or intra-population diversity. In some embodiments, generating molecules includes creating and/or modifying the molecules. In some embodiments, the fitness score is used to determine whether the two or more molecules: (1) are promising drug candidates; (2) are optimal to bind to a particular target; and/or (3) have properties that are likely to make them promising drug candidates. In some embodiments, the fitness score includes a diversity score.

In some embodiments, the generating the molecules of the subsequent populations includes chemically reasonable modifications so that, for example, the molecules are readily available to be synthesized. In yet other embodiments, the generating the molecules of the subsequent populations is based on reactions and corresponding reagents so that, for example, the molecules are readily available to be synthesized. In some embodiments, the molecule generated based on reactions and corresponding reagents is made of two or more synthons (e.g., reagents), wherein each synthon is associated with a particular reaction.

The methods described herein allow for more efficient searching to identify (e.g., generate) better diversity of molecules across different populations of molecules, wherein the identified molecules are just as likely, if not more likely, to exhibit desirable qualities as determined by one or more fitness scores, and wherein the identified molecules are readily available to be synthesized.

Systems, methods, and articles of manufacture, including computer program products, are provided for the generative design of small molecules. In some embodiments, a system includes at least one processor and at least one memory. The at least one memory may includes program code that provides operations when executed by the at least one processor.

In some embodiments, a computer program product includes a non-transitory computer readable medium storing instructions. The instructions may cause operations to be executed by at least one data processor.

In some embodiments, a system for generative design of small molecules includes at least one processor and at least one memory. The at least one memory may include program code that provides operations when executed by the at least one processor.

In some embodiments, a computer-implemented method comprises:
(b) obtaining, from a molecular structure database, an initial population of molecules;
(b) generating a first new population of molecules by modifying at least a first molecule in the initial population of molecules and selecting, from the initial population of molecules and at least the first modified molecule, a quantity of molecules satisfying one or more fitness scores;
(c) generating a second new population of molecules by modifying at least a second molecule in the first new population of molecules and selecting, from the first new population of molecules and at least the second modified molecule, another quantity of molecules satisfying the one or more fitness scores;
(d) in response to determining that a convergence criterion has not been satisfied, repeating step (c) at least once with the second new population of molecules as a first new population of molecules; and
(e) in response to determining that the convergence criterion has been satisfied, selecting a subset of the second new population of molecules as candidates for synthesis and testing.

In some embodiments, the generating the first new population of molecules of the method comprises (i) calculating one or more fitness scores for at least the first modified molecule and for each molecule of the initial population of molecules; and (ii) forming the first new population of molecules by selecting, from the initial population of molecules and at least the first modified molecule, the quantity of molecules having one or more fitness scores that satisfy the one or more fitness scores; and each instance of the generating the second new population of molecules comprises (i) calculating one or more fitness scores for at least the second modified molecule and each molecule in the first new population of molecules; and (ii) forming the second new population of molecules by selecting, from the first new population of molecules and at least the second modified molecule, the another N quantity of molecules having one or more fitness scores that satisfy the one or more fitness scores.

In some embodiments, the modifying each of the first molecule and the second molecule of the method comprises applying to the structure of the molecule one or more modifications selected from the group consisting of: substituting a non-hydrogen atom radical of the molecule with a moiety selected from a standard set of moieties; substituting a hydrogen atom of the molecule with a moiety selected from the standard set of moieties; replacing a divalent fragment of the molecule with a divalent moiety selected from the standard set of moieties, and wherein: a radical comprises from 1-5 non-hydrogen atoms or is a ring system comprising 3-10 non-hydrogen atoms; a moiety comprises from 1-5 non-hydrogen atoms or is a ring system comprising 3-10 non-hydrogen atoms; and a divalent fragment comprises from 1-5 non-hydrogen atoms or is a ring system comprising 3-10 non-hydrogen atoms.

In some embodiments, the one or more fitness scores comprise a diversity score. In some embodiments, the diversity score is indicative of a chemical similarity between pairs of molecules, and wherein the diversity score penalizes at least one molecule in a pair of molecules that are structurally or chemically similar to one another. In some embodiments, the diversity score is calculated from a metric selected from the group consisting of: a Tanimoto index, a cosine coefficient, a Dice metric, an Euclidean metric, a city-block metric, a Hamming index, and a Tversky index. In some embodiments, the one or more fitness scores comprises a calculated docking score for a molecule against a target binding site.

In some embodiments, the convergence criterion is selected from: a fixed number of repeats of step (c) of the method; an average fitness score that satisfies a threshold value, wherein the average fitness score is calculated for the second new population of molecules obtained from the last instance of step (c); and a designated number of molecules in the second new population of molecules obtained from the last instance of step (c) has a fitness score that is less than a threshold value.

In some embodiments, the obtaining the initial population of molecules of the method comprises: clustering, based on a similarity metric, a corresponding set of molecules in the molecular structure database into one or more clusters of molecules; and selecting, from each of the one or more clusters of molecules, one or more molecules having a fitness score satisfying one or more initial fitness scores.

In some embodiments, the similarity metric is a Tanimoto index, a cosine coefficient, a Dice metric, an Euclidean metric, a city-block metric, a Hamming index, or a Tversky index, and wherein the similarity metric is based on a property selected from: 2D similarity, 3D similarity, and a vector of physicochemical properties.

In some embodiments, the clustering is performed by applying one or more clustering algorithms selected from: Butina, centroid, CLink, Gower, McQuitty, SLink, Unweighted Pair Group Method with Arithmetic Mean (UPMGA), Ward, and Jarvis-Patrick.

In some embodiments, the one or more fitness scores for a molecule includes a calculated value of one or more of the following molecular properties: solubility, permeability, a selectivity score, an efficiency score, toxicity, and a physiologically based pharmacokinetic (PBPK) score.

In some embodiments, the initial population of molecules is obtained by a method chosen from: randomly selecting one or more molecules from the molecular structure database; selecting one or more molecules from the molecular structure database that have one or more physicochemical properties that meet threshold criteria; and selecting one or more molecules that have a particular scaffold; or a combination thereof.

Some embodiments of the method further comprise: (f) synthesizing at least one of the candidate molecules.

In some embodiments, the second new population of molecules is generated by selecting, as at least a portion of the another N quantity of molecules, a fixed proportion of molecules originating from the first new population of molecules.

In some embodiments, the one or more modifications for each molecule are identified by determining a set of applicable modifications for the molecule from a list of available modifications, and randomly choosing one or more modifications from the set of applicable modifications.

In some embodiments, a computer-implemented method comprises:
(a) obtaining, from a molecular structure database, a plurality of initial populations of molecules;
(b) generating a plurality of first new populations of molecules from each of the plurality of respective initial populations of molecules, wherein each of the plurality of first new populations of molecules comprises one or more molecules obtained by modifying one or more molecules in the initial population of molecules on which the first new population is based, wherein the molecules in each of the plurality of first new populations of molecules satisfy one or more fitness scores;
(c) generating a plurality of second new populations of molecules from each of the respective plurality of first new populations of molecules, wherein each of the plurality of second new populations comprise one or more molecules obtained by modifying one or more molecules in the first new population of molecules on which the second new population is based, wherein the molecules in each of the plurality of second new populations of molecules satisfy the one or more fitness scores;
(d) in response to determining that a convergence criterion has not been satisfied, repeating step (c) at least once with each of the first new populations of molecules being one of the second new populations of molecules obtained from the prior instance of step (c); and
(e) in response to determining that the convergence criterion has been satisfied, selecting a subset of molecules that comprises at least one molecule from each of the plurality of second new populations of molecules for synthesis and testing.

In some embodiments, the one or more fitness scores comprise a diversity score. In some embodiments, the diversity score is based on a structural similarity between a pair of molecules that comprises one molecule selected from each of two populations of molecules, and wherein the diversity score penalizes at least one molecule in a pair of molecules that are structurally similar to one another. In some embodiments, the diversity score is calculated from a metric selected from the group consisting of: a Tanimoto index, a cosine coefficient, a Dice metric, an Euclidean metric, a city-block metric, a Hamming index, and/or a Tversky index. In some embodiments, the diversity score is calculated between every pair of molecules that can be formed from molecules in one population and molecules in another population. In some embodiments, for any pair of molecules whose diversity score does not satisfy a threshold, only one molecule from the pair will satisfy the one or more fitness scores.

In some embodiments, the generating the plurality of first new population of molecules of the method comprises performing the following for each first new population of molecules: (i) calculating one or more fitness scores for at least the first modified molecule and for each molecule of the corresponding initial population of molecules; and (ii) forming the first new population of molecules by selecting, from the corresponding initial population of molecules and at least the first modified molecule, an N quantity of molecules having one or more fitnesss score that satisfy the one or more fitness scores; and each instance of the generating the plurality of second new population of molecules comprises performing the following for each second new population of molecules: (i) calculating one or more fitness scores for at least the second modified molecule and each molecule in the corresponding first new population of molecules; and (ii) forming the second new population of molecules by selecting, from the corresponding first new population of molecules and at least the second modified molecule, another N quantity of molecules having one or more fitness scores that satisfy the one or more fitness scores.

In some embodiments, a computer-implemented method comprises:
(a) obtaining an initial population of molecules wherein each molecule has a molecular structure constructed from a reaction database that contains a set of reactions, wherein each reaction from the set of reactions is associated with two or more sets of reagents, and wherein each molecule in the initial population of molecules is a product of a first reagent from a first set of reagents and a second reagent from a second set of reagents, and optionally a third reagent from a third set of reagents, in accordance with a reaction to which both the first and second reagents and the optional third set of reagents are associated;
(b) generating a first new population of molecules by
   modifying at least a first molecule in the initial population of molecules by replacing the first reagent from which the first molecule is formed with another first reagent from the first set of reagents associated with the reaction from which the first molecule is formed, and optionally replacing the second reagent from which the first molecule is formed with another second reagent from the second set of reagents associated with the reaction from which the first molecule is formed, thereby forming a first modified molecule, and
   selecting, from the initial population of molecules and at least the first modified molecule, a quantity of molecules that satisfy one or more fitness scores for inclusion in the first new population of molecules;
(c) generating a second new population of molecules by at least
   modifying at least a second molecule in the first new population of molecules by replacing the first reagent from which the second molecule is formed with another first reagent from the first set of reagents, and optionally replacing the second reagent from which the second molecule is formed with another second reagent from the second set of reagents thereby forming a second modified molecule, and
   selecting, from the first new population of molecules and at least the second modified molecule, another quantity of molecules that satisfy the one or more fitness scores;
(d) in response to determining that a convergence criterion has not been satisfied, repeating step (b) at least once with the second new population of molecules as a new initial population of molecules; and
(e) in response to determining that the convergence criterion has been satisfied, selecting a subset of the second new population of molecules as candidates for synthesis and testing.

In some embodiments, a computer-implemented method comprises:
(a) obtaining, from a molecular structure database, an initial population of molecules, each molecule of the initial population of molecules being a product of a first reaction between a first reagent selected from a first set of reagents associated with the first reaction and a second reagent selected from a second reagent selected from a second set of reagents associated with the first reaction, and optionally a third reagent from a third set of reagents associated with the first reaction;
(b) generating a first new population of molecules by
   creating a first modified molecule by applying a modification to at least a first molecule in the initial population of molecules, wherein the modification is selected from one or more of:
      replacing the first reagent from which the first molecule is formed with another first reagent selected from the first set of reagents;
      replacing the second reagent from which the first molecule is formed with another second reagent selected from the second set of reagents; and
      replacing a third reagent from which the first molecule is formed with another third reagent selected from the third set of reagents;
   and
   selecting, from the initial population of molecules and at least the first modified molecule, a quantity of molecules satisfying one or more fitness scores;
(c) generating a second new population of molecules by
   creating a second modified molecule by applying a modification to at least a second molecule in the second new population of molecules, wherein the modification is selected from one or more of:
      replacing the first reagent from which the first molecule is formed with another first reagent selected from the first set of reagents;
      replacing the second reagent from which the first molecule is formed with another second reagent selected from the second set of reagents; and
      replacing a third reagent from which the first molecule is formed with another third reagent selected from the third set of reagents;
   and
   selecting, from the first new population of molecules and at least the second modified molecule, another N quantity of molecules satisfying the one or more fitness scores;
(d) in response to determining that a convergence criterion has not been satisfied, repeating step (c) at least once with the second new population of molecules as a new initial population of molecules; and
(e) in response to determining that the convergence criterion has been satisfied, selecting a subset of the second new population of molecules as candidates for synthesis and testing.

In some embodiments, the generating a first new population of molecules of the method further comprises modifying an additional first molecule in the initial population of molecules by: selecting a third reagent that has a calculated similarity to the first reagent from which the additional first molecule is formed, wherein the third reagent is in an additional first set of reagents associated with an additional reaction, wherein the additional reaction is different to the reaction from which the additional first molecule is formed; constructing an additional first modified molecule by replacing the first reagent from which the additional first molecule is formed, with the third reagent, and replacing the second reagent from which the additional first molecule is formed with a fourth reagent selected from an additional second set of reagents associated with the additional reaction, wherein the fourth reagent has a calculated similarity to the second reagent; and selecting, from the initial population of molecules, the first modified molecule, and the additional first modified molecule, a quantity of molecules satisfying one or more fitness scores.

In some embodiments, the generating a second new population of molecules of the method further comprises modifying an additional second molecule in the initial population of molecules by: selecting a third reagent that has a calculated similarity to the first reagent from which the additional second molecule is formed, wherein the third reagent is in an additional first set of reagents associated with an additional reaction, wherein the additional reaction is different to the reaction from which the additional second molecule is formed; constructing an additional second modified molecule by replacing the first reagent from which the additional second molecule is formed with the third reagent, and replacing the second reagent from which the additional second molecule is formed, with a fourth reagent selected from a fourth additional second set of reagents associated with the additional reaction, wherein the fourth reagent has a calculated similarity to the second reagent; and selecting, from the first new population of molecules, the second modified molecule, and the additional second modified molecule, a quantity of molecules satisfying one or more fitness scores.

In some embodiments, a system comprises at least one data processor; and at least one memory storing instructions, which when executed by the at least one data processor, result in operations comprising any of the methods described herein.

In some embodiments, a non-transitory computer readable medium stores instructions, which when executed by at least one data processor, result in operations comprising any of the methods described herein.

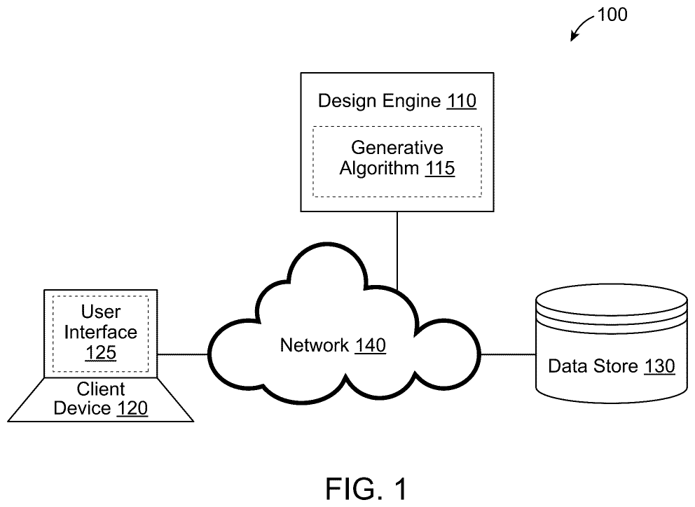

FIG. 1 depicts a system diagram that illustrates an example of a small molecule design system 100, in accordance with some example embodiments. Referring to FIG. 1, the small molecule design system 100 may include a design engine 110, a client device 120, and a data store 130. As shown in FIG. 1, the design engine 110, the client device 120, and the data store 130 may be communicatively coupled via a network 140. The client device 120 may be a processor-based device including, for example, a workstation, a desktop computer, a laptop computer, a smartphone, a tablet computer, a wearable apparatus, and/or the like. The data store 130 may be a database, such as a molecular structure database, implemented as, for example, a relational database, a graph database, an in-memory database, a non-SQL (NoSQL) database, a filesystem, and/or the like. The network 140 may be a wired network and/or a wireless network including, for example, a local area network (LAN), a virtual local area network (VLAN), a wide area network (WAN), a public land mobile network (PLMN), the Internet, and/or the like.

Instead of an indiscriminate search of the vast chemical space of possibly relevant molecules, a design engine is configured to apply a generative algorithm in which one or more new populations of molecules are generated based on one or more initial populations of molecules, and each new population is assessed for its properties. In some embodiments, the generative algorithm is a genetic algorithm. In some embodiments, the design engine 110 may be configured to apply a generative algorithm 115 to generate, based on one or more initial populations of molecules, and through one or more iterations, one or more subsequent populations of molecules. In some cases, the one or more initial populations of molecules and the one or more subsequent populations of molecules may include small molecules having a molecular weight from approximately 100 Daltons to approximately 1,000 Daltons. Moreover, in some cases, at least one initial population of molecules may include a random selection of molecules from a set of molecules (e.g., a set of all known molecules such as available from Chemical Abstracts Service or a medicinal chemistry database, or a subset of the set of all known molecules, pre-filtered for particular drug-like properties).

For each iteration of the generative algorithm, the design engine may modify one or more molecules from an initial population of molecules before selecting, from a pool of the initial population of molecules and the one or more modified molecules, a number of molecules that exhibit certain desirable properties as indicated by one or more fitness scores. In some embodiments, the modification of the one or more molecules is a chemically reasonable modification. Examples of approaches in which molecules are modified at each generation are further described herein. One example of a fitness score is a docking score against a target of interest. In the context of molecular design and modeling, a docking score is a measure of the binding affinity between two molecules after the two molecules have undergone a docking process (e.g., shape complementarity analysis, docking simulation, and/or the like) to predict the preferred orientation of the two molecules when the two molecules are bound to form a stable complex. Other examples of fitness scores include one or more of a diversity score, a solubility score, a permeability score, a selectivity score, an efficacy score, a toxicity score, and a physiologically based pharmacokinetic (PBPK) score. For example, by evaluating fitness based on measures such as strength of docking against a target, solubility, permeability, selectivity, efficacy, toxicity, physiologically based pharmacokinetics (PBPK), the chance of obtaining suitable molecules is increased. Strength of docking against a target can be calculated or estimated by any of a number of docking methods, algorithms, or programs. Accordingly, the molecules selected to form the new population of molecules may include molecules from the initial population of molecules determined to exhibit the desirable properties as well as modified molecules determined to exhibit the desirable properties. This selection strategy for forming each new population of molecules preferably maximizes the likelihood that the best performing molecules from each population of molecules, including those that may not have undergone any modification, survive on to subsequent iterations of the generative algorithms. Subsequent populations of molecules are generated by modifying the best performing molecules from previous populations of molecules.

In this way, the design engine performs fitness evaluation and selection based on complete molecules, and not fragments of molecules, or molecules constructed by capping frameworks or core structures with simple groups such as methyl or phenyl, the design engine is able to ensure the relevance of the fitness scores to the target in question.

In some embodiments, the design engine may perform multiple iterations of the generative algorithm. In some embodiments, the design engine generates the one or more new populations of molecules based on a single initial population of molecules, whereas in other embodiments, multiple initial populations of molecules may be optimized separately from one another and in parallel. The design engine continues to generate additional new populations of molecules until one or more convergence conditions are satisfied, at which point one or more molecules from the new populations of molecules may be synthesized and tested, or identified as candidates for further review prior to synthesis and testing.

The one or more convergence conditions may require that the fitness scores of a certain quantity of molecules included in the one or more new populations of molecules satisfy a threshold or a convergence criterion. For example, the fitness scores for the molecules exhibit a below-threshold improvement over the fitness scores of other populations of molecules (e.g., generated based on the same initial population of molecules or a different initial population of molecules). Where fitness scores for more than one property are calculated, the fitness scores for the respective properties may be weighted according to adjustable parameters that reflect the relative importance of those properties. In some embodiments, convergence is determined by docking scores of the population of molecules whereas other fitness scores are used to filter out molecules having one or more properties that are less desirable.

In some embodiments, the design engine may impose one or more limitations on the modifications made to each molecule when generating a new population of molecules, to further increase the likelihood that the molecules included in the new population of molecules exhibit the same desirable properties as those in the previous populations of molecules. For example, the modifications made to each molecule may be limited to a fixed number of modifications to ensure that the structures in one generation do not depart significantly in form from the structures in the prior generation.

In some embodiments, the modifications made to each molecule are limited to one or more chemically reasonable modifications, i.e., modifications that would be reasonable to an organic chemist. An exemplary approach to identifying a set of such modifications is described in Polischchuk, et al., J. Cheminform., 12:28, 1-18, (2020), incorporated herein by reference.

A molecule typically comprises one or more fragments, each of which can be defined as being an atom, or a group of atoms that are bonded to one another, and located at a defined attachment point between the fragment and the remainder of the molecule. Varying definitions of fragment are consistent with the methods herein. Accordingly, a chemically reasonable modification of a molecule includes replacing one fragment of the molecule, defined in this manner, with another fragment. For example, concepts of isosterism can be employed to limit modifications to replacing one fragment with another fragment that is of a comparable size. Thus, a pyridyl group could be a chemically reasonable modification of a phenyl group because the two are isosteric. Additionally, properties of a fragment such as polarity can be utilized: so a chemically reasonable modification of a hydroxyl group could be an amino group or a thio group. In instances where a certain portion of the molecule is designated as a core or a scaffold, the modifications made to the molecule may exclude changes to the core or scaffold itself, and be thereby limited to variations of fragments attached to the scaffold. For example, a molecule that contains a fused heterocyclic ring system such as indole can be modified in such a way that only substituents on the indole moiety are identified as fragments and are modified and the indole moiety is itself maintained in all molecules based on the original molecule.

In some embodiments, the types of fragments that are changed are selected from lists of functional groups that are familiar to chemists, and can be illustrated by, but not limited to, the following examples: single atom fragments include halogen atoms; two-atom fragments that attach via one of the two atoms include groups such as hydroxyl, cyano, and thio. Three-atom fragments that attach via one of the three atoms include amino, nitro, and carbenyl. Four-atom fragments that attach via a specified atom include: carboxylic acid, sulfonate, and methyl. In each category, a list of fragments can be drawn up and utilized as a standard set, from which fragments can be arbitrarily chosen or selected according to some criterion or criteria, such as size or polarity. In instances where the lists of known functional groups prove to be too limited, a more expansive definition can be employed: for example, a fragment can be defined to identify one or more attachment point(s) between the fragment and the remainder of the molecule, and include an atom at each of the one or more attachment points and those one or more other atoms that are within a certain radius of that atom. Radius can be defined geometrically, such as a through-space threshold distance when a 3D model of the fragment is available, or by a number of bonds separating the attachment point from the other atoms in the fragment. Additionally, fragments can be defined to include rings or ring systems, such as: cyclopropyl, phenyl, indolyl, and others.

In some instances, the chemically reasonable modifications made to a molecule may be selected from a group that includes: substituting a non-hydrogen atom radical of the molecule with a moiety selected from a standard list of moieties; substituting a hydrogen atom of the molecule with a moiety selected from the standard list of moieties; and replacing a divalent fragment of the molecule with a divalent moiety selected from the standard list of moieties. In this context, a radical may include from 1-5 non-hydrogen atoms or be a ring system comprising 3-10 non-hydrogen atoms. A moiety may include from 1-5 non-hydrogen atoms or is a ring system comprising 3-10 non-hydrogen atoms. A divalent fragment may include from 1-5 non-hydrogen atoms, or is a ring system comprising 3-10 non-hydrogen atoms. The term radical is understood to mean a fragment that includes an unpaired electron or “dangling bond” at its forthcoming point of attachment; correspondingly the site of a molecule to which it will be attached contains a “dangling bond” at the point at which a fragment will be attached. The use of the term radical herein is one of convenience because the fragments themselves do not represent isolatable molecules.

In some embodiments, the manner of creating molecules and modifying them at each generation is based on reactions and corresponding reagents, rather than fragments. Reactions correspond to well-understood chemical transformations (e.g., alcohol reacts with carboxylic acid to form an ester) where each reagent admits of variation. For example, in an esterification reaction, the alcohols can be selected from alkyl alcohols, (methanol, ethanol, propanol, etc.) and cycloalkyl alcohols (cyclopropanol, cyclobutanol, etc.). A two-component reaction is based on two sets of reagents (e.g., alcohols and acids) that can combine in a particular way. Reactions can also be three-component reactions, in which case three sets of reagents are available for choosing each of the components. The methods herein are generalizable to four-component and even five-component reactions, even though such schemes are rare in actual chemistry.

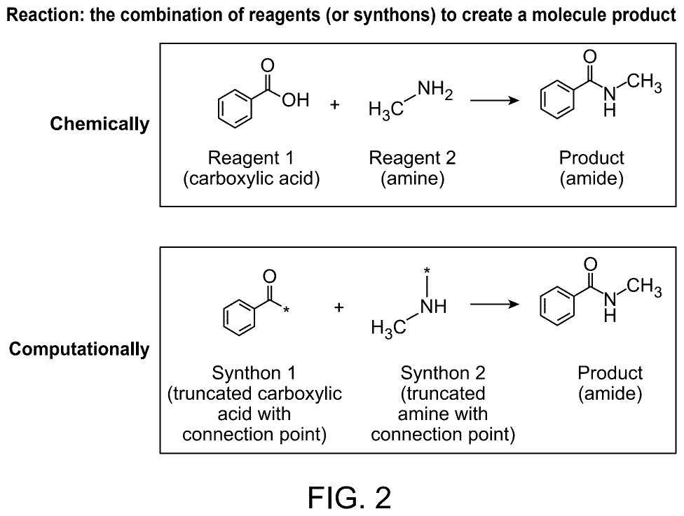

FIG. 2 illustrates combining two reagents to form a molecule product, both chemically and as encoded computationally. In the upper panel of FIG. 2, an amide forming reaction between benzoic acid and methylamine is shown. In this scheme, reagent 1 (benzoic acid) has been selected from a list of carboxylic acids, and reagent 2 (methylamine) has been chosen from a second list, of amines. In the actual reaction, a hydrogen atom attached to the amino group is “lost” because it combines with the hydroxyl group in the acid to form H₂O. The formation of “byproduct” water is not of interest when creating amide molecules computationally. Computationally, such reagents are represented as “synthons” in which a connection point of a reagent to a counterpart reagent for a given reaction is identified, in place of a fragment comprising one or more atoms. In the lower panel of FIG. 2, the amine and acid reagents are illustrated as computationally stored “synthons”. For convenience, computationally, the H and OH groups from the respective amino and carboxylate moieties are deleted from the reagent molecules when forming their representative synthons. The reagents are stored in truncated form as “synthons” wherein their points of attachment are identified as capable of forming bonds with the respective points of attachment on other synthons. Each individual synthon is therefore a building block within a molecule that represents a starting reagent in a synthesis of that molecule.

Accordingly, each molecule in the initial population of molecules can be constructed as a product of a reaction between a first reagent selected from a first set of reagents associated with the reaction and a second reagent selected from a second set of reagents associated with the reaction, and optionally (in the case of a 3-component reaction) a third reagent selected from a third set of reagents associated with the reaction. A population will typically comprise molecules constructed from a number of different reactions, each of which has its own sets of reagents from which molecules can be built. For example, an amine and a carboxylic acid react to form an amide. For an amide-forming reaction, then, molecules can be formed by selecting from a first list of amines as first reagents, and a second list of carboxylic acids as second reagents. A population can comprise molecules formed by an amide-forming reaction in addition to molecules formed from an esterification reaction.

Accordingly, the modifications made to each molecule when generating a new population of molecules may include changing at least one of the first reagent, the second reagent, and, where applicable, the third reagent, for a given reaction. In cases where the modifications include a change to the first reagent, the modifications may be limited to replacing the first reagent with another first reagent selected from the first set of reagents associated with the reaction. In cases where the modifications additionally include a change to the second reagent, the modifications to the second reagent may be limited to replacing the second reagent with another second reagent selected from the second set of reagents associated with the reaction. When constructing an initial population of molecules, the reactions may be selected, for example, randomly, from a canonical set of reactions. Moreover, in some cases, each of the first and second reagents associated with a given reaction may also be selected, for example, in a random fashion, from corresponding sets of first and second reagents.

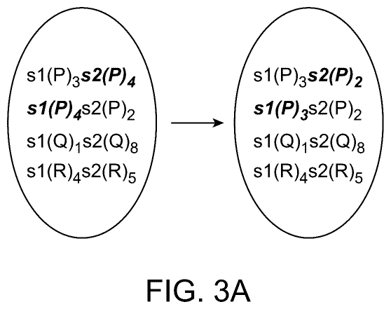

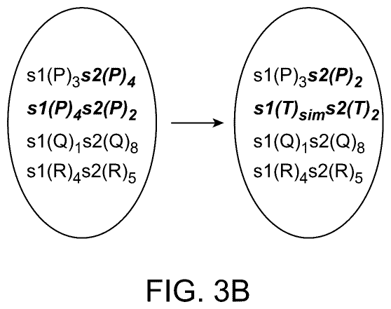

A general description of modifying molecules by replacing one or more reagents from which they are formed is illustrated in FIG. 3A. The notation sn(X); means that the molecule is formed by reaction X. For a reaction X that requires 2 or more reagents, synthon sn(X)ᵢ denotes the nth reagent (n=1, 2, 3, etc.), and the ith member of the set of nth reagents. Thus, s1(X)₄ means the 4^{th} synthon from the first set of synthons (the first reagent) for reaction X. On the left hand side of FIG. 3A are 4 representative molecules in a population: all of the molecules are formed from two components, s1 and s2; the first two molecules were formed from reaction P; the third and fourth were formed from reactions Q and R, respectively. In forming the next generation population, the third and fourth molecules are unchanged (due to their overall calculated fitness, as further described herein). The first and second molecules are both transformed though the underlying reaction (P) from which they are both formed is not changed. In the first molecule, only the second reagent is changed (to another reagent chosen from a list of second reagents), whereas in the second molecule only the first reagent is changed (to another reagent chosen from a list of first reagents). It should be noted that, in going from s1(P)₃s2(P)₄ to s1(P)₃s2(P)₂, although only the second reagent has changed, the reagent s2(P)₂ must be selected from the set of second reagents that can perform reaction P.

In a further embodiment, a general description of modifying molecules by replacing both the reaction and one or more reagents is illustrated in FIG. 3B, in which the notation is the same as in FIG. 3A. In FIG. 3B, the starting population on the left hand side is the same as the starting population in FIG. 3A. In the embodiment of FIG. 3B, the first, third, and fourth molecules proceed to the next generation population in the same manner as with FIG. 3A. For the second molecule, formed from a first reagent s1(P)₄ and a second reagent s2(P)₂ according to a first reaction (P), the modification includes identifying a new first reagent s1(T)ₛᵢₘ that has a minimal threshold similarity to the first reagent s1(T)₄ but which is utilized in a second reaction (T) that is different from the first reaction (P). The second reaction (T) relies on a second set of first reagents and a second set of second reagents from (P). Accordingly a modified molecule is now constructed by using the second reaction between the new first reagent s1(T)ₛᵢₘ=and a new second reagent (s2(T)₂ in FIG. 3B selected from the second set of second reagents associated with reaction (T). In other embodiments, not shown, the new second reagent may also be selected to have at least a minimal similarity to the second reagent that was used to form the initial molecule. In FIG. 3B, then, it is illustrated that the next generation of molecules comprises at least some molecules that pass through unmodified, at least some molecules that are modified according to the embodiment of FIG. 3A, and at least some molecules that are modified according to the way in which the second molecule is modified. It would be further understood that the new population can further comprise other molecules built from reaction (T) as well as still other molecules built from other reactions, (U), (V), etc. The similarity between reagents utilized in the embodiment of FIG. 3B may be according to a Tanimoto coefficient based on atom composition or based on shape similarity or some other suitable method as described elsewhere herein.

In some embodiments, application of the generative algorithm 115 includes more than one iteration in which a population of molecules is generated. In a first such iteration, the design engine 110 modifies at least a first molecule from an initial population of molecules. Furthermore, a single iteration during application of generative algorithm 115 includes the design engine 110 selecting, from the initial population of molecules and at least the first modified molecule, a quantity of molecules for inclusion in a new population of molecules based on one or more fitness scores. Examples of fitness scores may include a diversity score, a docking score against a target, a solubility score, a permeability score, a selectivity score, an efficacy score, a toxicity score, and a physiologically based pharmacokinetic (PBPK) score. Accordingly, the molecules selected to form the new population of molecules may exhibit certain desirable properties as indicated by the fitness scores of the molecules. In doing so, the design engine 110 may maximize the likelihood that the best performing molecules from each population of molecules survive onto subsequent iterations of the generative algorithm 115. Subsequent populations of molecules formed in this manner may include molecules generated by modifying the best performing molecules from previous populations of molecules and are thus more likely to exhibit the same desirable properties.

The design engine 110 may cause the generative algorithm 115 to perform one or more iterations and generate the one or more new populations of molecules based on a single initial population of molecules. Alternatively and/or additionally, the design engine 110 may cause the generative algorithm 115 to perform one or more iterations and generate the one or more new populations of molecules based on multiple different initial populations of molecules. In some cases, the design engine 110 may cause the generative algorithm to continue to generate additional new populations of molecules until one or more conditions are satisfied. Another example condition may include the design engine 110 having generated a certain quantity of molecules whose fitness scores satisfy one or more thresholds. Other examples of conditions may include the fitness scores for the new population of molecules satisfying a threshold or a convergence criterion in which the fitness scores for the new population of molecules exhibit an improvement over the fitness scores of other populations of molecules (e.g., generated based on the same initial population of molecules or a different initial population of molecules). Such an improvement may be quantified as being below a pre-defined value (threshold) of a particular quantity.

Once the one or more conditions are satisfied, at least one molecule from the one or more new populations of molecules may be synthesized and tested or identified as a candidate for synthesis and testing. In some cases, the molecule that is synthesized and tested or identified as a candidate for synthesis and testing may be determined as a result of the design engine 110 applying one or more filters or threshold criteria. Moreover, in some instances, the design engine 110 may generate, for display at the client device 120, a user interface 125 providing a visual representation of the molecules identified as candidates for synthesis and testing.

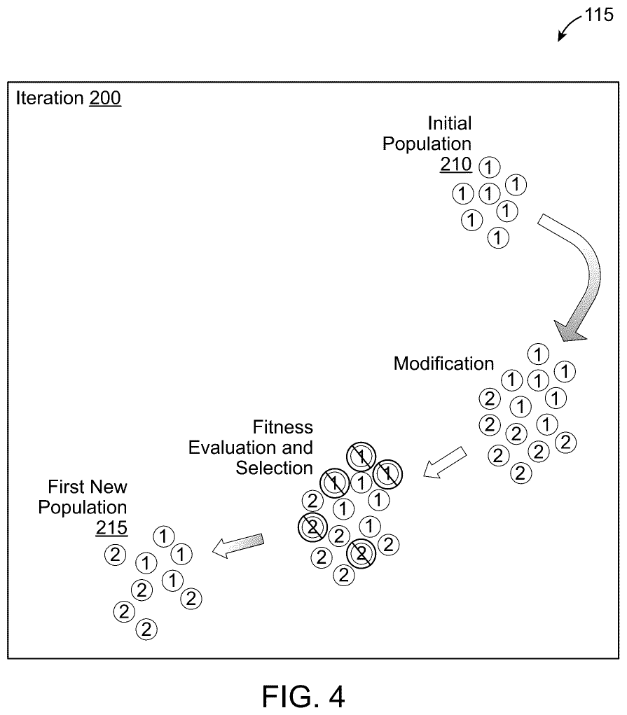

To further illustrate, FIG. 4 depicts a single iteration 200 of an example of the generative algorithm 115 for small molecule design. As shown in FIG. 4, the single iteration 200 of the generative algorithm 115 may include starting with an initial population 210 of molecules (denoted as circles marked with the number “1” in FIG. 4). In some cases, the initial population 210 of molecules may be obtained from the data store 130 through random selection, selection of one or more molecules exhibiting one or more physicochemical properties that meet threshold criteria, selection of one or more molecules having a particular scaffold, and/or the like. Typically the initial population of molecules consists of a number, N, of molecules chosen by a user. N may be a number such as 500, 1,000, 2,000, 5,000, or greater depending on the computing resources available. The behavior and operation of the algorithm herein does not depend on the size of the initial population.

The generative algorithm includes modifying at least a first molecule from the initial population 210 to generate at least the one or more first modified molecules (denoted as circles marked with the number “2” in FIG. 4). As explained elsewhere herein, a given molecule may be susceptible to more than one modification, so that more than one distinct modified molecule may arise from a step of applying single modifications to that molecule.

In some instances, a portion of the first molecule from the initial population 210 of molecules, may be designated as core portion of the first molecule. Such a core portion is typically a scaffold or a framework to which various fragments are attached. For example, the core portion of the first molecule may be associated with certain desirable properties, in which case the modifications made to the first molecule should exclude changes to the core portion of the first molecule.

Accordingly, when modifying the first molecule, the design engine 110 may avoid making changes to the core portion of the first molecule. Avoiding making changes to the core portion may increase the likelihood that the desirable properties of the first molecule are retained when modifications are made to it.

In some example embodiments, each molecule from the initial population 210 of molecules may be a product of a reaction between a first reagent selected from a first set of reagents associated with the first reaction and a second reagent selected from a second set of reagents associated with the first reaction. In some cases, the first reagent and the second reagent may be represented as synthons, or individual building blocks, selected from a corresponding set of synthons associated with the reaction. Accordingly, to modify the first molecule from the initial population 210 of molecules, in one embodiment, the design engine 110 may change at least one of the first reagent, and the second reagent that form the first molecule. For example, in cases where the modification includes changing the first reagent, the modification may be limited to replacing the first reagent in the first molecule with another first reagent selected from the first set of reagents associated with the reaction. In cases where the modification includes additionally changing the second reagent, the modification may be limited to replacing the second reagent in the first molecule with another second reagent selected from the second set of reagents associated with the reaction.

It should be understood that any one molecule in the initial population of molecules may be subject to more than one modification and therefore may give rise to more than one distinct modified molecule. For example, a given molecule may be modified in 2 or more different ways, corresponding to two or more chemically reasonable modifications (such as changing two or more different fragments). In another example, a molecule built from two reagents according to a particular reaction can be modified two more times (thereby creating two or more modified molecules), because either of the two reagents can independently be replaced, and each reagent can be replaced separately by more than one reagent from a set of reagents.

It should also be understood that more than one molecule in the initial population of molecules 210 will typically be subject to their own respective one or more modifications.

The initial population 210 of molecules and at least the first modified molecule then undergo fitness evaluation and selection in order to form a first new population 215 of molecules. For example, in some cases, the design engine 110 may select, from the initial population 210 of molecules and at least the first modified molecule, a quantity of molecules based at least on a first set of fitness scores associated with each molecule in the initial population 210 of molecules and at least the first modified molecule. For example, a quantity of molecules may be selected for inclusion in the first new population 215 of molecules wherein each molecule has a fitness score that satisfies one or more thresholds. Alternatively, to generate the first new population 215 of molecules, the design engine 110 may select a quantity of molecules having a highest fitness score. In some cases, the quantity of molecules may be selected by at least comparing a respective fitness score of one or more randomly selected pairs of molecules from the initial population 210 of molecules and at least the first modified molecule and including, in the first new population 215 of molecules, a molecule having a higher fitness score from each pair of molecules. In some cases, the quantity of molecules selected for inclusion in the first new population 215 of molecules may include a fixed proportion of molecules from the first initial population 210 of molecules.

The first new population of molecules may be fixed to have the same number, N, of molecules as the initial population of molecules. This ensures that the overall population of molecules under consideration does not dwindle away yet does not expand in a manner that reduces overall efficiency of the algorithm.

Because the initial population 210 of molecules and at least the first modified molecule undergo a fitness evaluation as part of the selection process, the resulting first new population 215 of molecules is preferably populated by molecules exhibiting certain desirable properties. For example, the fitness scores for a molecule in the first new population may include one or more of a diversity score, a docking score against a target, a solubility score, a permeability score, a selectivity score, an efficacy score, a toxicity score, and a physiologically based pharmacokinetic (PBPK) score. Accordingly, the molecules selected for inclusion in the first new population 215 of molecules may exhibit certain desirable properties with respect to diversity, target binding affinity, solubility, permeability, selectivity, efficacy, toxicity, and physiological based pharmacokinetics (PBPK).

In cases where certain molecules from the initial population 210 of molecules already exhibit the desirable properties, those molecules may survive the selection process and be included in the first new population 215 of molecules to undergo, in some cases, one or more additional iterations of the generative algorithm 115. For instance, the example of the first new population 215 of molecules shown in FIG. 4 includes molecules from the initial population 210 of molecules (denoted as circles marked with the number “1”) as well as molecules generated by modifying molecules from the initial population 210 of molecules (denoted as circles marked with the number “2” in FIG. 4).

In some embodiments, the design engine 110 may impose one or more diversity criteria (e.g., diversity scores) when forming the first new population 215 of molecules in order to ensure that the composition of the population is not dominated by multiple molecules that have similar structures to one another, and thereby optimize the exploration of chemical space. For example, exploration of chemical space may be increased by maximizing intra-population diversity within the first new population 215 of molecules. As used herein, the term “intra-population diversity” may refer to a dissimilarity (e.g., structural and/or chemical dissimilarity) between the molecules included in a population of molecules. Accordingly, to maximize intra-population diversity, the design engine 110 may cluster the initial population 210 of molecules and at least the first modified molecule into one or more clusters of similar molecules (e.g., structurally and/or chemically similar molecules). In some cases, the design engine 110 may apply a recognized clustering algorithm (e.g., Butina, centroid, Clink, Gower, McQuitty, Slink, Unweighted Pair Group Method with Arithmetic Mean (UPMGA), Ward, Jarvis-Patrick, and/or the like) in order to cluster the molecules based on a diversity score (e.g., a Tanimoto index, a Dice index, a cosine coefficient, a Soergel distance, a Euclidean metric, a city-block metric, a Hamming index, a Tversky index, and/or the like) of each molecule. Accordingly, a first molecule in a first cluster may be similar to a second molecule in the first cluster but dissimilar to a third molecule from a second cluster. Thus, upon generating the one or more clusters of similar molecules, the design engine 110 may select, from each cluster of similar molecules, one or more molecules having a highest fitness score (e.g., as calculated elsewhere herein) for inclusion in the first new population 215 of molecules. For instance, the one or more molecules selected for inclusion in the first new population 215 of molecules may be associated with one or more of a highest docking score against a target, solubility score, permeability score, a selectivity score, efficacy score, toxicity score, and physiologically based pharmacokinetic (PBPK) score. Accordingly, the resulting first new population 215 of molecules may include molecules that exhibit certain desirable properties and are diverse relative to one another.

Referring again to FIG. 4, in some example embodiments, the design engine 110 may increase the likelihood that the molecules included in the first new population 215 of molecules exhibit properties that are comparable to the properties of the molecules included in the initial population 210 of molecules by imposing one or more limitations on the types of modifications made to any molecule in the initial population 210 of molecules. For example, the design engine 110 may limit the modifications made to a molecule in the initial population 210 of molecules to those selected from a set of chemically reasonable modifications, as further described elsewhere herein.

Additionally, in order to limit the number of modified molecules to a practical number, the design engine 110 may limit the number of modifications made to a molecule in the initial population 210 of molecules to a threshold number of modifications, such as 1, 2, or 3.

In preferred embodiments, the design engine 110 performs multiple iterations of the generative algorithm 115 to generate multiple successive new populations of molecules after starting from a single initial population of molecules. Accordingly, the new population of molecules that is generated by performing one iteration of the generative algorithm 115 is then used as a starting population of molecules for performing a subsequent iteration of the generative algorithm 115. That is, each successive iteration of the generative algorithm 115 is performed using a population of molecules that corresponds to the new population of molecules generated by a previous iteration of the generative algorithm 115.

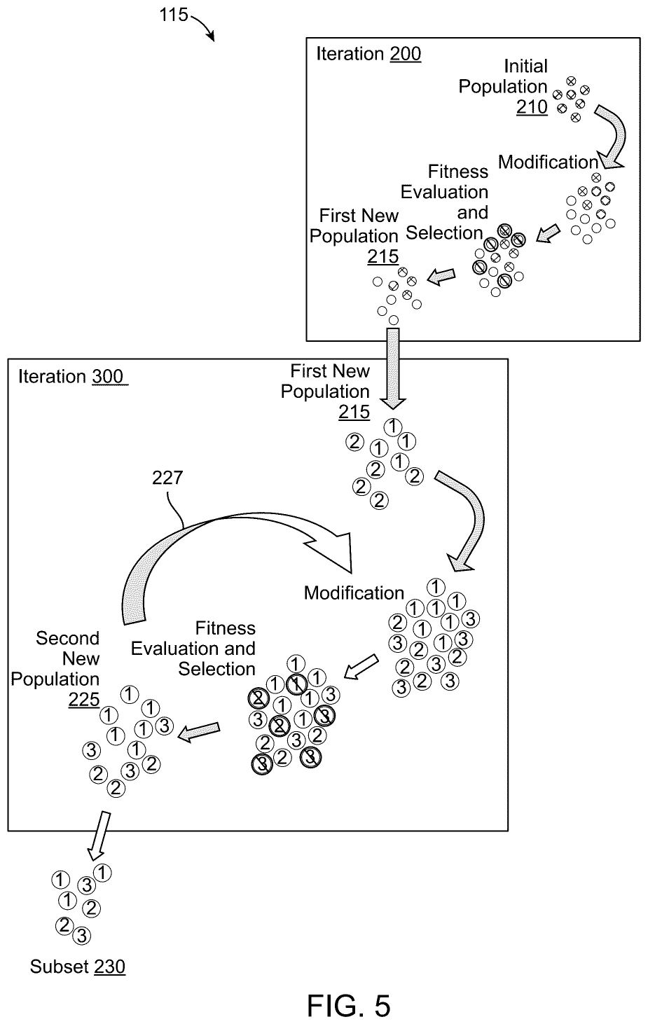

To further illustrate, FIG. 5 depicts an example of the generative algorithm 115 for small molecule design in which multiple iterations are performed. Referring to FIG. 5, in which iteration 200 is recapitulated from FIG. 4, the design engine 110 performs two or more additional iterations of the generative algorithm 115 subsequent to performing iteration 200. Whereas iteration 200 of the generative algorithm 115 is performed based on the initial population 210 of molecules, a subsequent iteration 300 of the generative algorithm 115 is performed based on the first new population 215 of molecules. The first time that iteration 300 is performed, the first new population 215 of molecules was generated by iteration 200. On subsequent iterations 227, the second new population becomes the first new population to which modifications and fitness evaluation are applied.

Referring again to FIG. 5, iteration 300 of the generative algorithm 115 includes the design engine 110 modifying at least one molecule from the first new population 215 of molecules to generate one or more second modified molecules (denoted as circles marked with the number “3” in FIG. 5). (As shown in FIG. 4, since the first new population 215 of molecules may include molecules from the initial population 210 of molecules as well as molecules formed by modifying molecules from the first initial population 210 of molecules, the modifying may now be applied to any such molecule, regardless of whether it originated in the initial population, or arose as a modified molecule, both of which were selected for inclusion in the first new population.)

To generate a second new population 225 of molecules, the design engine 110 selects, from the first new population 215 of molecules and at least the one or more second modified molecules, a number of molecules based on a second set of fitness scores associated with each molecule in the first new population 215 of molecules and at least the one or more second modified molecules. For example, a molecule may be selected for inclusion in the second new population 225 of molecules if the molecule has a fitness score that satisfies one or more thresholds. Alternatively, the design engine 110 may select a number of molecules having a highest fitness score for inclusion in the second new population 225 of molecules. For instance, in some cases, the design engine 110 may select the number of molecules by at least comparing a respective fitness score of one or more randomly selected pairs of molecules from the first new population 215 of molecules and at least the second modified molecule, and including, in the second new population 225 of molecules, a molecule having a higher fitness score from each pair of molecules. In some cases, the quantity of molecules selected for inclusion in the second new population 225 of molecules may include a fixed proportion of molecules originating from the first new population 215 of molecules.

As shown in FIG. 5, after a particular convergence criterion is reached, the design engine 110 further selects, for example, by applying one or more filters and threshold criteria, one or more molecules from the second new population 225 to form a first subset 230 of molecules. In some cases, the first subset 230 of molecules is identified as candidates for synthesis and testing.

In some example embodiments, to further maximize the exploration of the vast chemical space, the design engine may also impose one or more diversity criteria (e.g., diversity scores) when generating one or more new populations of molecules that derive from a same initial population of molecules or from different initial populations of molecules. For example, to ensure diversity amongst successive new populations of molecules generated based on the same initial population of molecules, the fitness scores determining whether a molecule is selected for inclusion in the subset of molecules may include a diversity score (e.g., a Tanimoto index, a Dice index, a cosine coefficient, a Soergel distance, a Euclidean metric, a city-block metric, a Hamming index, Jaccard index, a Tversky index, and/or the like) indicative of a structural and/or chemical similarity between the molecule and each molecule in the initial population of molecules. Thus, in some embodiments, the design engine 110 may impose one or more diversity criteria (e.g., diversity scores) when generating the second new population 225 of molecules in order to maximize intra-population diversity within the second new population 225 of molecules as well as inter-population diversity between the second new population 225 of molecules and one or more previous populations of molecules such as the initial population 210 of molecules and the first new population 215 of molecules. To maximize intra-population diversity within the second new population 225 of molecules, the design engine 110 may cluster the first new population 215 of molecules and at least the second modified molecule, for example, by applying a clustering algorithm as further described herein, into one or more clusters of similar molecules (e.g., structurally and/or chemically similar molecules). The design engine 110 may then select, from each cluster of similar molecules, one or more molecules having a highest fitness score for inclusion in the second new population 225 of molecules.

As used herein, the term “inter-population diversity” may refer to a dissimilarity (e.g., structural and/or chemical dissimilarity) between the molecules included in different populations of molecules. Accordingly, to maximize inter-population diversity, the design engine 110 may select the second new population 225 of molecules based on a diversity score of each molecule included in the first new population 215 of molecules and at least the second modified molecule. In some instances, the diversity score of a molecule may be a metric, such as a Tanimoto index, a Dice index, a cosine coefficient, a Soergel distance, a Euclidean metric, a city-block metric, a Hamming index, a Tversky index, and/or the like, indicative of a similarity (e.g., structural and/or chemical similarity) between the molecule and each molecule in the first new population 215 of molecules and at least the second modified molecule. By imposing this diversity criteria (e.g., diversity scores), the design engine 110 may ensure the most dissimilar (e.g., structurally and/or chemically dissimilar) molecules are selected for inclusion in the second new population 215 of molecules. This is advantageous because, as will be described in more detail below, the design engine 110 may also impose the diversity criteria (e.g., diversity scores) to maximize inter-population diversity between the second new population 225 of molecules and populations of molecules generated by other iterations of the generative algorithm 115 performed using one or more different initial populations of molecules.

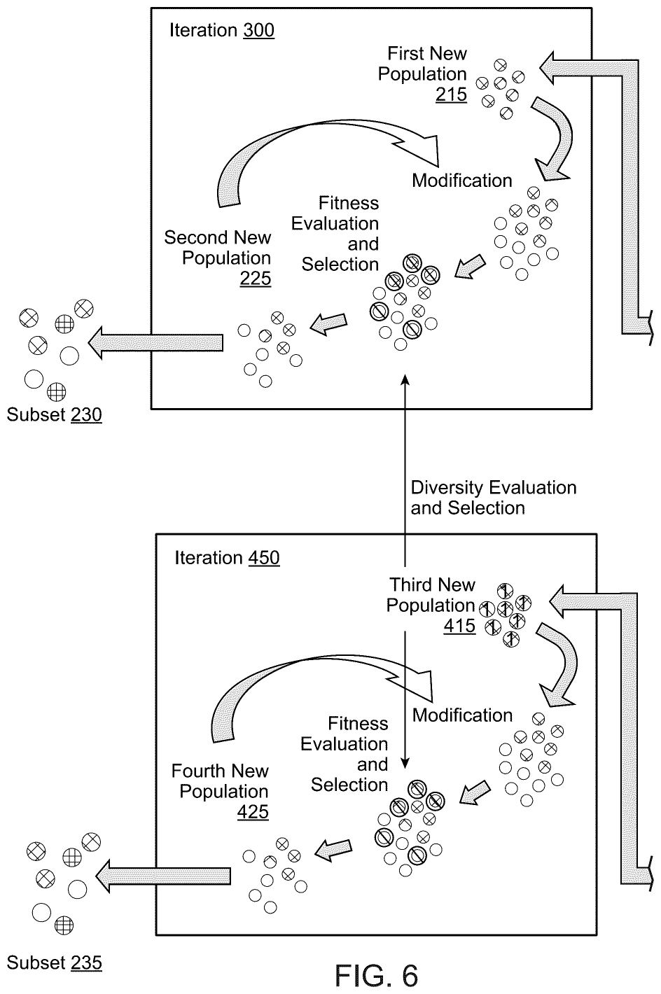

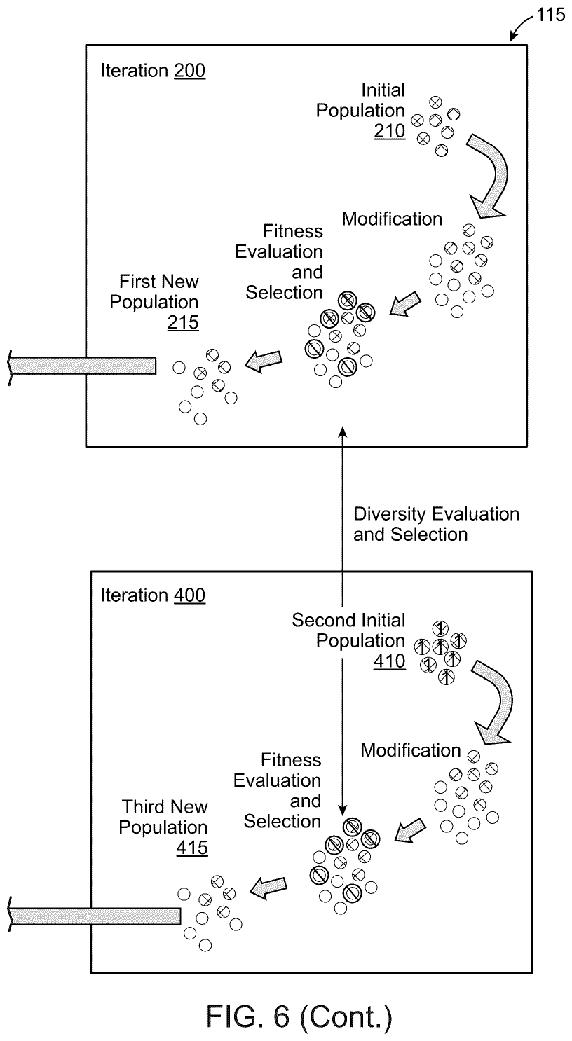

In another embodiment, the design engine 110 performs multiple iterations of the generative algorithm 115 to generate multiple subsequent populations of molecules from multiple respective different initial populations of molecules. FIG. 6 depicts four iterations of the generative algorithm 115 with two of the four iterations each being performed on two different respective initial populations of molecules. Subsequent iterations of the generative algorithm 115 applied to the second new population 225 and the fourth new population 425 to generate subsequent new population for each of the population 225 and 425, respectively, are illustrated by the curved left-to-right pointing arrows. In some embodiments, not illustrated here, the design engine 110 performs multiple iterations of the generative algorithm 115 on three or more initial populations of diverse molecules to generate three or more new populations of molecules with certain characteristics. For example, FIG. 6 shows that, in addition to performing at least one iteration of the generative algorithm 115 based on a first initial population of molecules 210 (e.g., the iteration 200 and the subsequent iteration 300), the design engine 110 may also perform one or more iterations of the generative algorithm 115 based on a second initial population 410 of molecules that is different than the first initial population 210 of molecules. Second initial population 410 may be obtained, for example, from the data store 130. In some embodiments, one or more molecules of second initial population 410 are modified as described herein, and one or more of the modified or unmodified molecules are selected based on a diversity score with respect to the modified or unmodified molecules of the first initial population 210 (as depicted by the vertical arrow between these two populations). In some embodiments, a modified molecule of the second initial population 410 is only selected if the diversity score exceeds a certain threshold value. For the iteration 400 of the generative algorithm 115, for instance, the design engine 110 may modify one or more molecules from the second initial population 410 of molecules before selecting, from the second initial population 410 of molecules and the one or more modified molecules, a quantity of molecules based on a third set of fitness scores associated with each molecule in the second initial population 410 of molecules and the one or more modified molecules. In doing so, the design engine 110 generates a third new population 415 of molecules. In the example shown in FIG. 6, the design engine 110 continues to perform another iteration 450 of the generative algorithm 115 based on the third new population 415 of molecules.

For instance, the iteration 450 of the generative algorithm 115 may include the design engine 110 modifying one or more molecules in the third new population 415 of molecules before selecting, from the third new population 415 of molecules and the one or more modified molecules, another quantity of molecules satisfying one or more fitness criteria (e.g., fitness scores) for inclusion in a fourth new population 425 of molecules.

Furthermore, to ensure diversity amongst subsequent populations of molecules generated from different initial populations of molecules, the fitness score that determines whether a molecule is selected for inclusion in the subset of molecules may include another diversity score (e.g., a Tanimoto index, a Dice index, a cosine coefficient, a Soergel distance, a Euclidean metric, a city-block metric, a Hamming index, Jaccard index, a Tversky index, and/or the like) indicative of a chemical and/or structural similarity between the molecule and each molecule in a different initial population of molecules and/or a population of molecules generated by iterations of the generative algorithm 115.

Accordingly, in some example embodiments, the design engine 110 may impose one or more diversity criteria (e.g., diversity scores) to maximize inter-population diversity between the third new population 415 of molecules and the populations of molecules generated by other iterations of the generative algorithm 115 performed using one or more different initial populations of molecules. In the example shown in FIG. 6, the third new population 415 of molecules may be selected from the second initial population 410 of molecules and one or more molecules generated by modifying molecules from the second initial population 410 of molecules based on a diversity score associated with each molecule. In some embodiments, a molecule of the modified or unmodified (i.e., initial) molecules for the third new population 415 is only selected if the diversity score exceeds a certain threshold value. For example, modifications quite different from those applied in iteration 200 to generate modified molecules of the initial population 210 are more likely to result in molecules selected for the third new population 415, because the molecules'diversity score with respect to modified molecules of the initial population 210 exceeds a certain threshold value. This diversity score may be a metric, such as a Tanimoto index, a Dice index, a cosine coefficient, a Soergel distance, a Euclidean metric, a city-block metric, a Hamming index, a Tversky index, and/or the like, indicative of a similarity (e.g., structural and/or chemical similarity) between two or more molecules. Similarly, the design engine 110 may select the fourth new population 425 of molecules based on a second diversity score associated with the molecules in the third new population 415 of molecules and the molecules generated by modifying one or more molecules in the third new population 415 of molecules. This second diversity score may also be a metric, such as a Tanimoto index, a Dice index, a cosine coefficient, a Soergel distance, a Euclidean metric, a city-block metric, a Hamming index, a Tversky index, and/or the like, indicative of a similarity (e.g., structural and/or chemical similarity) between two or more molecules.

As noted, the design engine 110 may perform one or more iterations of the generative algorithm 115 to generate one or more subsequent populations of molecules, such as the first new population 215 of molecules, the second new population 225 of molecules, the third new population 415 of molecules, and the fourth new population 425 of molecules. That each subsequent population of molecules is generated to include molecules having satisfactory fitness scores ensures the same desirable properties are preserved and propagated through each successive generation of molecules. Meanwhile, by penalizing molecules that are too similar (e.g., having a diversity score below a certain threshold), thereby maximizing intra-population and/or inter-population diversity, ensures that the molecules included in each subsequent population of molecules are novel and diverse (e.g., structurally and/or chemically dissimilar) relative to one another as well as to molecules in other populations of molecules. As shown in FIG. 6, in some cases, the design engine 110 may select, from the fourth new population 425 of molecules, a second subset 235 of molecules as candidates for synthesis and testing.

Maximizing the exploration of the chemical space (or a certain portion thereof) may also be achieved, for example, by imposing one or more diversity criteria (e.g., diversity scores) to maximize intra-population and inter-population diversity across successive generations of molecules generated by the generative algorithm 115, may further ensure that the molecules generated by the generative algorithm 115 are novel relative to previously known molecules.

### Example

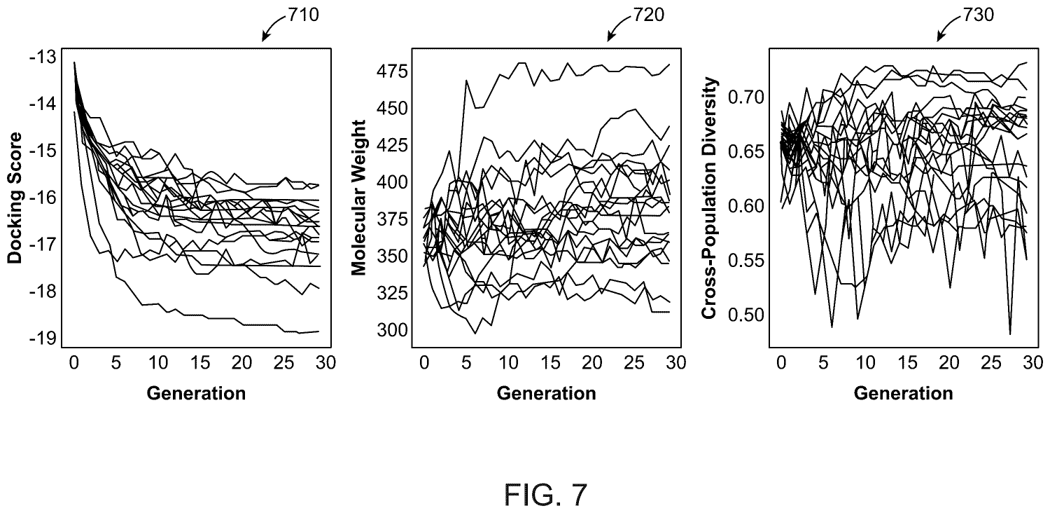

To further illustrate, FIG. 7 depicts graphs illustrating the evolution of 3 respective molecular properties across about 20 populations of molecules evolved by performing multiple iterations of the generative algorithm 115, optimized against the same target. In this optimization scheme, the scoring function is a weighted combination of the docking score, and the diversity score. According to the diversity score, similarity of pairs of molecules is scored as a penalty.

The three panels of FIG. 7 each depict progression of a particular molecular property, as successive generations of 20 populations of molecules are created and tested for fitness by calculation of various properties. In a given panel, each line represents a particular run of the algorithm, as successive generations starting from an initial population are created; the particular value at each generation represents an average of the property in question over the molecules in that population that have the best values of the property. As shown in FIG. 7, graph 710, each successive generation of molecules generated by the application of the generative algorithm 115 exhibits a successively improving target binding affinity as indicated by the decreasing average docking scores of the molecules. (Docking scores are represented as a logio of a computed binding energy on an arbitrary energy scale. The more negative is the energy of binding, the better a molecule binds to the target.) However, while the docking improves overall, each successive generation of molecules exhibits significant structural variation, as indicated by the average molecular weights of the molecules in a given population in panel 720, and the diversity scores of the populations in graph 730.

The main advantage of the algorithm is that the populations are intrinsically diverse. Most populations have diversity scores of >0.5 when compared to other populations, meaning that the molecules are structurally diverse from other molecules in the other populations. In panel 730, each line represents the average distance of molecules in the population from all the other populations.

A diversity score of 0.2-0.3 would represent a significant lack of diversity. Most populations have a diversity in the 0.7-0.8 range, representing a very high amount of diversity.

Fluctuations in diversity occur during the time that the algorithm runs because it utilizes a generative algorithm which doesn't guarantee anything about smooth convergence. Furthermore, the convergence criterion is principally governed by the properties of the individual molecules (such as a docking score). Hence there is also a competition going on between maintaining low (good) docking scores and high diversity. Sometimes this competition leads to sizeable fluctuations in diversity because a large improvement in one category can lead to a disruption to the performance of the other.

### Example—Positive Control

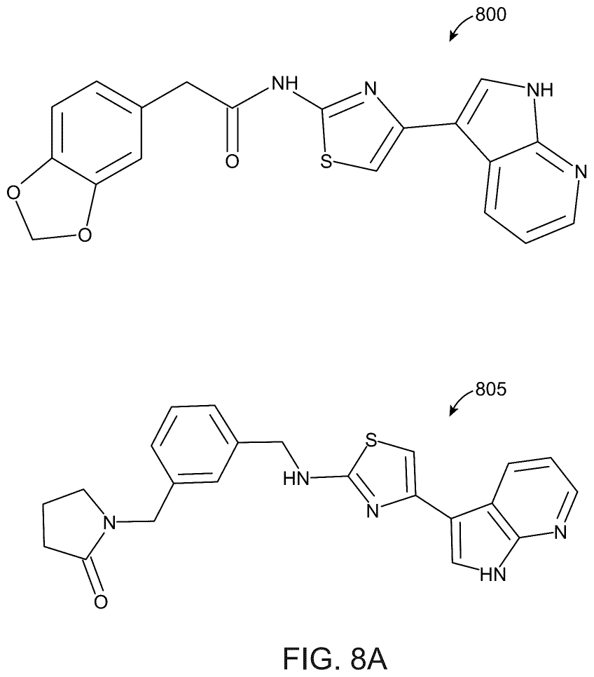

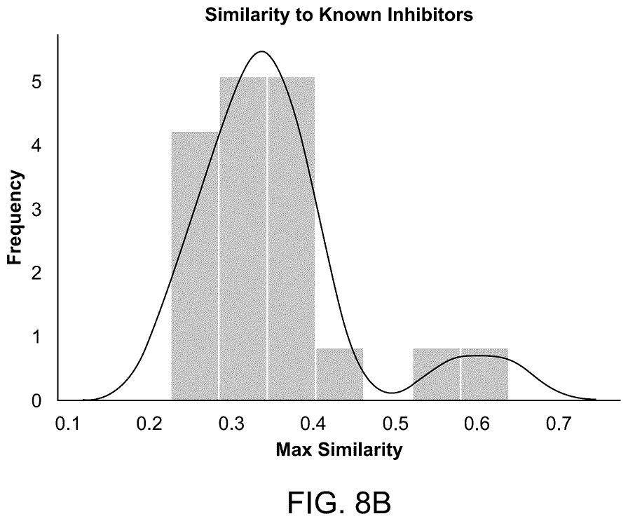

The performance of generative algorithm 115 may be further assessed in terms of various benchmarks. FIG. 8A depicts one type of benchmark, which evaluates whether the molecules (e.g., a first molecule 805) generated by the generative algorithm 115 (when starting from a random selection of starting molecules) are structurally similar to known molecules (such as molecule 800) that are already established to have a desirable property (e.g., an inhibitor capable of binding and blocking the activity of the same target as the first molecule). Molecules 800 and 805, depicted in FIG. 8A, are very similar to one another.

### Example

In another example benchmark, we quantify the novelty of the molecules generated by the generative algorithm 115. This is done based on calculating a similarity metric between 20 successive generations of the molecules and certain known nearest inhibitors of the target in question (in this case ROCK1 Kinase) from ChEMBL. Such a similarity metric can be, for example, Tanimoto index, Dice index, cosine coefficient, Soergel distance, a Euclidean metric, a city-block metric, a Hamming index, a Tversky index, and/or the like.

FIG. 8B depicts the distribution of nearest neighbor similarities between molecules in each of the 20 populations and the known molecules. Each bar in the histogram is number of populations whose average similarity (for a particular population w.r.t. the ‘known’ inhibitors) is in a particular range of similarity (0.200-0.299; 0.300-0.399, etc.) The continuous line superimposed on the figure is simply to guide the eye. Most of the populations have average overall similarities falling in the 0.2-0.4 Tanimoto range, suggesting low similarity to existing inhibitors (i.e., a greater likelihood of novelty). But there are 1-2 populations in the 0.5-0.7 range, suggesting a higher similarity to known inhibitors. The summary from this plot is that the algorithms herein can generate molecules that are both structurally distinct from already known inhibitors as well as molecules that are highly similar to known inhibitors. That is, the molecules generated by the generative algorithm 115 are both novel and have a high likelihood of exhibiting the same desirable property as previously known molecules (such as those inhibitors for ROCK1 Kinase that are identified in ChemBL).

### Example

Another important benchmark is to evaluate whether application of the generative algorithm 115 adequately explores the chemical space of drug-like molecules. Because the algorithm herein does not exhaustively sample all possible compounds in a given space, we want to know how well it does compared to an exhaustive enumeration and docking of all possible compounds. So the goal of this example is to compare the results from the algorithm herein to those obtained from docking all possible compounds in a particular space. Such a comparison is not possible for a database of ˜10¹⁰ available molecules, so a subspace is chosen for which docking of all molecules is possible.

Accordingly, in this example, a small subset of a much larger, but available, chemical space was evaluated. In practice this subset comprised around 10⁶ randomly chosen molecules from a database of ˜10¹⁰ available molecules (in this case found in the REadily Accessible (REAL) chemical space (see Internet site enamine.net/compound-collections/real-compounds/real-space-navigator), though other chemical databases would suffice). Each of those molecules in the randomly chosen subset was docked against a target. The outcome of those dockings is referred to as the “ground truth” from the point of view of the scoring function for the molecules (in this case a docking score). We identified the 5 top docking molecules from a rank-ordered list of 10⁶ molecules that were docked.

For comparison purposes, the starting point of an application of the generative algorithm herein are random compounds selected from the subspace of 10⁶ molecules. The reactions that are used to form those molecules are used as starting points in an application of the algorithm, so that further molecules are formed by varying the reagents that can react according to those reactions, and/or by varying the reactions (and corresponding reagents) themselves. After iterating to convergence, we show that the algorithm generates and recovers 4 out of the top 5 molecules that were obtained from the random subset of molecules.

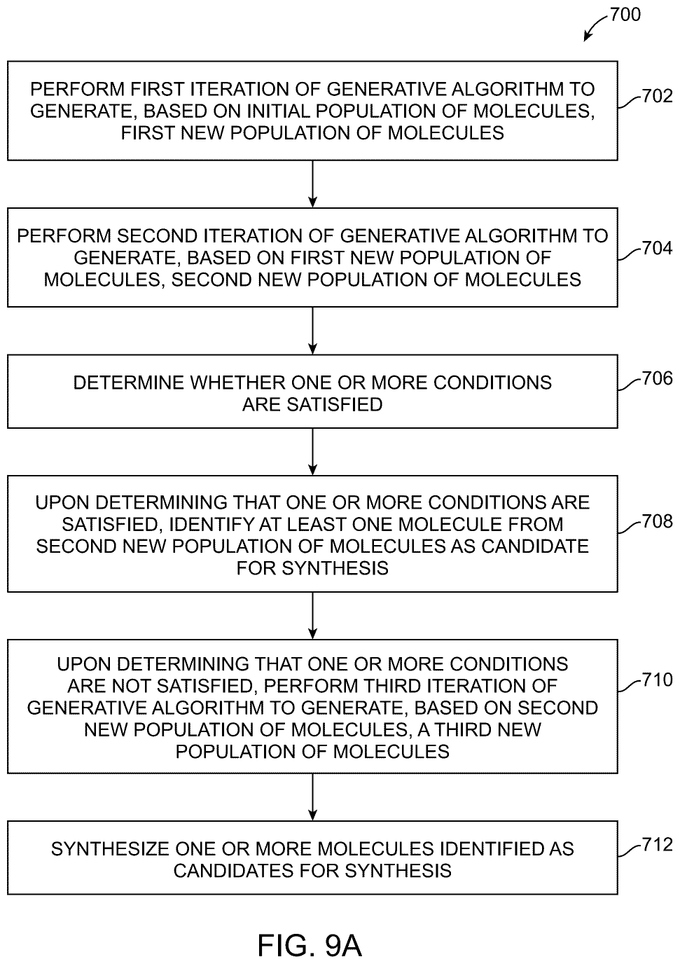

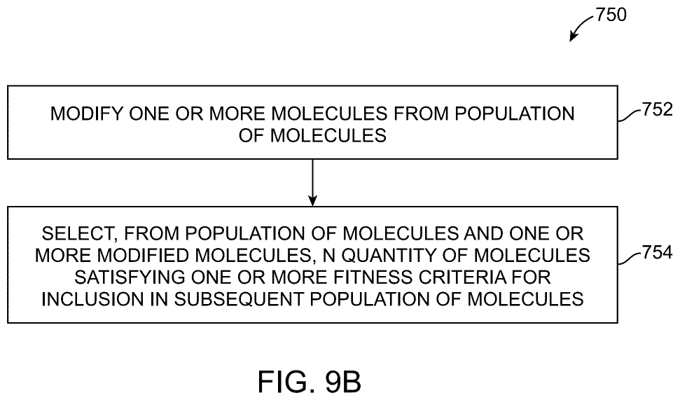

FIG. 9A depicts a flowchart illustrating an example of a process 700 for designing small molecules. Referring to FIGS. 1 and 9A, the process 700 may be performed by the design engine 110 to generate one or more molecules, such as small molecules having a molecular weight from approximately 100 Daltons to approximately 1,000 Daltons, for synthesis or as candidates for synthesis.

At 702, the design engine 110 performs a first iteration of the generative algorithm 115 to generate, based on an initial population of molecules, a first new population of molecules. For example, as shown in FIG. 4, the design engine 110 may perform the first iteration 200 of the generative algorithm 115 to generate, based on the first initial population 210 of molecules, the first new population 215 of molecules. As shown in FIG. 4, the iteration 200 of the generative algorithm 115 may include the design engine 110 modifying one or more molecules from the first initial population 210 of molecules. Moreover, as shown in FIG. 4, the iteration 200 of the generative algorithm 115 may include the design engine 110 selecting, from the first initial population 210 of molecules and the one or more modified molecules, a quantity, N, of molecules that satisfy one or more fitness criteria (e.g., fitness scores) for inclusion in the first new population 215 of molecules.

At 704, the design engine 110 may perform a second iteration 300 of the generative algorithm 115 to generate, based on the first new population of molecules 215, a second new population of molecules 225. As shown in FIG. 5, the design engine 110 may generate the second new population 225 of molecules by modifying one or more molecules from the first new population 215 of molecules and selecting, from the first new population 215 of molecules and the one or more modified molecules, another N quantity of molecules satisfying the one or more fitness criteria (e.g., fitness scores) for inclusion in the second new population 225 of molecules.

At 706, the design engine 110 may determine whether one or more conditions are satisfied. In some example embodiments, the design engine 110 may continue to perform additional iterations of the generative algorithm 115 until one or more conditions are satisfied. One example condition may include the design engine 110 having generated a threshold quantity of subsequent new populations of molecules. Another example condition may include the design engine 110 having identified a threshold quantity of molecules satisfying the one or more fitness criteria (e.g., fitness scores). Other examples of conditions may include the fitness scores for the first new population 215 of molecules and/or the second new population 225 of molecules satisfying a threshold or one or more convergence criteria in which the fitness scores exhibit a below-threshold improvement over the fitness scores of other populations of molecules (e.g., generated based on the same initial population 210 of molecules or a different initial population of molecules).

At 708, upon determining that the one or more conditions are satisfied, the design engine 110 may identify at least one molecule from the second new population of molecules as a candidate for synthesis. For example, if the design engine 110 determines that the one or more conditions are satisfied, the design engine 110 may select, for example, the subset 230 of molecules from the second new population 225 of molecules as candidates for synthesis and testing. In some cases, the design engine 110 may select the subset 230 of molecules by applying one or more filters or threshold criteria.

At 710, upon determining that the one or more conditions are not satisfied, the design engine 110 may perform a third iteration of the generative algorithm 115 to generate, based on the second new population of molecules or a second initial population of molecules, a third new population of molecules. In some example embodiments, where the design engine 110 determines that the one or more conditions are not satisfied, the design engine 110 may perform one or more additional iterations of the generative algorithm 115 to generate one or more additional new populations of molecules.

At 712, the design engine 110 identifies one or more molecules, from the new population of molecules that satisfies the one or more convergence conditions, as candidates for synthesis and/or testing. For example, the molecules that are synthesized or identified as candidates for synthesis and/or testing may be determined by the design engine 110 applying one or more filters or threshold criteria. In preferred embodiments, the one or more molecules identified as candidate for synthesis and/or testing may undergo synthesis.

In other preferred embodiments, the one or more molecules identified as candidate for synthesis and/or testing may undergo testing. The certain molecules identified as candidates for synthesis and/or testing can be tested, such as screened for particular measured physicochemical properties as well as additional calculated and predicted properties, as a further filtering step before more labor intensive tests such as pre-clinical testing.

FIG. 9B shows steps of a process 750 that correspond to a single iteration of the generative algorithm 115 performed by the design engine 110, for example, in operations 702, 704, and 710 of the process 700 described with respect to FIG. 9A.

At 752, the design engine 110 may modify one or more molecules from a given population of molecules. For instance, in the example of the iteration 200 shown in FIG. 4, the design engine 110 may generate the first new population 215 of molecules by modifying one or more molecules from the first initial population 210 of molecules. To increase the likelihood that the molecules included in the subsequent population of molecules exhibit the same desirable properties as those in the previous population of molecules, the design engine 110 may limit the modifications made to each molecule to, for example, one or more chemically reasonable mutations. In other instances, where a molecule is a product of a first reaction between a first reagent selected from a first set of reagents associated with the first reaction and a second reagent selected from a second set of reagents associated with the first reaction, the design engine 110 may modify the molecule by changing one or more of the first reaction, the first reagent, and the second reagent.

At 754, the design engine 110 may select, from the population of molecules and the one or more modified molecules, a quantity of molecules satisfying one or more fitness criteria (e.g., fitness scores) for inclusion in a subsequent population of molecules. As further described elsewhere herein, the quantity of molecules may be selected for inclusion in the subsequent population of molecules based on each molecule satisfying one or more fitness criteria (e.g., fitness scores) with respect to, for example, diversity, docking against a target, solubility, permeability, selectivity, efficacy, toxicity, physiologically based pharmacokinetic properties (PBPK), and/or the like.

### Computational Implementations

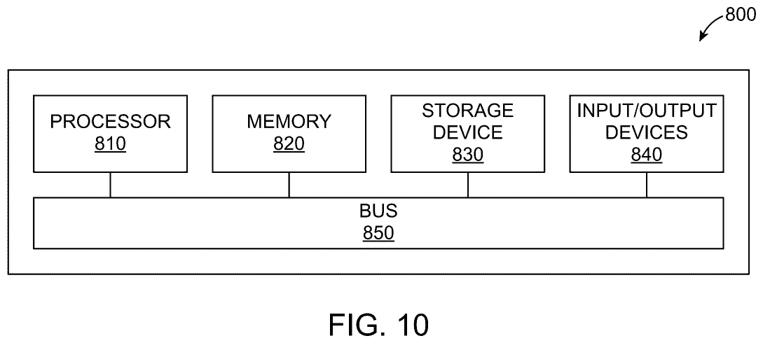

FIG. 10 depicts a block diagram illustrating an example of a computing system 800 consistent with implementations of the current subject matter. Referring to FIGS. 1 and 10, the computing system 800 may incorporate the design engine 110 and/or any components therein.

As shown in FIG. 10, the computing system 800 can include one or more processors 810, a memory 820 which will typically include both high speed random access memory as well as non-volatile memory (such as one or more magnetic disk drives), one or more storage device(s) 830, and one or more input/output device(s) 840. The processor 810, the memory 820, the storage device 830, and the input/output device 840 can be interconnected via a system bus 850.

The one or more processors 810 are each capable of processing instructions for execution within the computing system 800. Such executed instructions can implement one or more components of, for example, the design engine 110. In some implementations of the current subject matter, the processor 810 can be a single-threaded processor. Alternately, the processor 810 can be a multi-threaded processor. The processor 810 is capable of processing instructions stored in the memory 820 and/or on the one or more storage device(s) 830 to display output such as graphical information for a user interface provided via the one or more input/output device(s) 840.

The memory 820 is a computer readable medium such as volatile or non-volatile that stores information within the computing system 800. The memory 820 can store data structures representing molecular structures, molecular properties, and chemical reactions, for example.

The one or more storage device(s) 830 are capable of providing persistent storage for the computing system 800. The storage device 830 can be a hard disk device, an optical disk device, or a removable device such as a floppy disk device, or a tape device, or other suitable persistent storage means.

The one or more input/output device(s) 840 provides input/output functions for the computing system 800. In some implementations of the current subject matter, the one or more input/output device(s) 840 include one or more of a keyboard, a pointing device, as well as other manners of providing user-inputs. In various implementations, the one or more input/output device(s) 840 includes a display unit for displaying graphical user interfaces.

According to some implementations of the current subject matter, the one or more input/output device(s) 840 can provide input/output operations for a network device. For example, the one or more input/output device(s) 840 can include Ethernet ports or other networking ports to communicate with one or more wired and/or wireless networks (e.g., a local area network (LAN), a wide area network (WAN), the Internet). As such, the input/output devices can communicate with network 140 and one or more client devices 120.

The results of the calculations (such as population diversity) created by the technology herein, as well as the generated molecular structures themselves, can be displayed in tangible form, such as on one or more computer displays, such as a monitor, laptop display, or the screen of a tablet, notebook, netbook, or cellular phone. The numerical results can further be printed to paper form, stored as electronic files in a format for saving on a computer-readable medium or for transferring or sharing between computers, or projected onto a screen of an auditorium such as during a presentation.

In some implementations of the current subject matter, the computing system 800 can be used to execute various interactive computer software applications that can be used for organization, analysis and/or storage of data in various (e.g., tabular) format (e.g., Microsoft Excel®, and/or any other type of software). Alternatively, the computing system 800 can be used to execute any type of software applications. These applications can be used to perform various functionalities, such as planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 840. The user interface can be generated and presented to a user by the computing system 800 (e.g., on a computer screen monitor).

As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens, with or without a stylus, or other touch-sensitive devices such as single or multi-point resistive or capacitive track pads, voice recognition hardware and software, optical scanners, optical pointers, digital image capture devices and associated interpretation software, a speech-recognition device, gesture-recognition technology, human fingerprint reader, or other input such as based on a user's eye-movement, and the like.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

The methods described herein are preferably implemented as computer programs, executed by one or more computer systems, and the implementation is within the capability of those skilled in the art. In particular, the computer functions for manipulations of molecular structures, stored digitally, can be developed and implemented by a programmer skilled in the art. These computer programs, which can also be referred to as programs, software, software applications, applications, components, or code, include machine instructions for a programmable processor, and can be implemented in a number and variety of programming languages including, in some cases, mixed implementations (i.e., relying on separate portions written in more than one computing language suitably configured to communicate with one another). For example, the programs, as well as any required scripting functions, can be programmed in a number of compiled or non-compiled languages, including but not limited a high-level procedural and/or object-oriented programming language, and/or in assembly/machine language. Such languages can be selected from: C, C++, Java, JavaScript, VisualBasic, Tcl/Tk, Python, Perl, .Net languages such as C #, and other equivalent languages. The capability of the technology is not limited by or dependent on the underlying programming language used for implementation or control of access to the basic functions. Alternatively, the functionality could be implemented from higher level functions such as tool-kits that rely on previously developed functions for manipulating bit-strings and fingerprints.

To the extent that a given implementation relies on other software components, already implemented, such as functions for calculating molecular similarity functions, reading molecular databases, and calculating closeness of fit of a molecular structure of a protein active site, those functions can be assumed to be accessible to a programmer of skill in the art.

In the case of generating a random number, or a pseudo-random number, the process is preferably not solely based on a mathematical formula or process. Preferably the choice of a random number, or the seed for a random number generation method, is obtained from a fluctuating quantity in the real world, such as an electrical potential within the computing device being employed.

The methods of the present technology may also draw upon functions contained in one or more dynamically linked libraries, not shown in FIG. 10, but stored either in memory 820, or on disk 830.

The manner of operation of the technology, when reduced to an embodiment as one or more software modules, functions, or subroutines, can be in a batch-mode-as on a stored database of molecular structures, processed in batches, or by interaction with a user who inputs specific instructions for a single molecular structure.

Various implementations of the technology herein can be contemplated, particularly as performed on computing apparatuses of varying complexity, including, without limitation, workstations, PC's, laptops, notebooks, tablets, netbooks, and other mobile computing devices, including cell-phones, mobile phones, and personal digital assistants. The computing devices can have suitably configured processors, including, without limitation, graphics processors and math coprocessors, for running software that carries out the methods herein. In addition, certain computing functions are typically distributed across more than one computer so that, for example, one computer accepts input and instructions, and a second or additional computers receive the instructions via a network connection and carry out the processing at a remote location, and optionally communicate results or output back to the first computer.

Finally, it is to be understood that the executable instructions that cause a suitably-programmed computer to execute methods for generative design of molecular structures, as described herein, can be stored and delivered in any suitable computer-readable format. This can include, but is not limited to, a portable readable drive, such as a large capacity “hard-drive”, or a “pen-drive”, such as connects to a computer's USB port, and an internal drive to a computer, and a CD-Rom or an optical disk. It is further to be understood that while the executable instructions can be stored on a portable computer-readable medium and delivered in such tangible form to a purchaser or user, the executable instructions can be downloaded from a remote location to the user's computer, such as via an Internet connection which itself may rely in part on a wireless technology such as WiFi. Such an aspect of the technology does not imply that the executable instructions take the form of a signal or other non-tangible embodiment. The executable instructions may also be executed as part of a “virtual machine”implementation.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A computer-implemented method, comprising:
(a) obtaining, from a molecular structure database, an initial population of molecules;
(b) generating a first new population of molecules by modifying at least a first molecule in the initial population of molecules and selecting, from the initial population of molecules and at least the first modified molecule, a quantity of molecules satisfying one or more fitness scores;
(c) generating a second new population of molecules by modifying at least a second molecule in the first new population of molecules and selecting, from the first new population of molecules and at least the second modified molecule, another quantity of molecules satisfying the one or more fitness scores;
(d) in response to determining that a convergence criterion has not been satisfied, repeating step (c) at least once with the second new population of molecules as a first new population of molecules; and
(e) in response to determining that the convergence criterion has been satisfied, selecting a subset of the second new population of molecules as candidates for synthesis and testing.

2. The method of claim 1, wherein:
the generating the first new population of molecules comprises:
(i) calculating one or more fitness scores for at least the first modified molecule and for each molecule of the initial population of molecules; and
(ii) forming the first new population of molecules by selecting, from the initial population of molecules and at least the first modified molecule, the quantity of molecules having one or more fitness scores that satisfy the one or more fitness scores;
and
each instance of the generating the second new population of molecules comprises:
(i) calculating one or more fitness scores for at least the second modified molecule and each molecule in the first new population of molecules; and
(ii) forming the second new population of molecules by selecting, from the first new population of molecules and at least the second modified molecule, the another N quantity of molecules having one or more fitness scores that satisfy the one or more fitness scores.

3. The method of claim 2, wherein the modifying each of the first molecule and the second molecule comprises:
applying to the structure of the molecule one or more modifications selected from the group consisting of: substituting a non-hydrogen atom radical of the molecule with a moiety selected from a standard set of moieties; substituting a hydrogen atom of the molecule with a moiety selected from the standard set of moieties; replacing a divalent fragment of the molecule with a divalent moiety selected from the standard set of moieties, and wherein:
a radical comprises from 1-5 non-hydrogen atoms or is a ring system comprising 3-10 non-hydrogen atoms;
a moiety comprises from 1-5 non-hydrogen atoms or is a ring system comprising 3-10 non-hydrogen atoms; and
a divalent fragment comprises from 1-5 non-hydrogen atoms or is a ring system comprising 3-10 non-hydrogen atoms.

4. The method of claim 2, wherein the one or more fitness scores comprise a diversity score.

5. The method of claim 4, wherein the diversity score is indicative of a chemical similarity between pairs of molecules, and wherein the diversity score penalizes at least one molecule in a pair of molecules that are structurally or chemically similar to one another.

6. The method of claim 4, wherein the diversity score is calculated from a metric selected from the group consisting of: a Tanimoto index, a cosine coefficient, a Dice metric, an Euclidean metric, a city-block metric, a Hamming index, and a Tversky index.

7. The method of claim 2, wherein the one or more fitness scores comprises a calculated docking score for a molecule against a target binding site.

8. The method of claim 1, wherein the convergence criterion is selected from:
a fixed number of repeats of step (c);
an average fitness score that satisfies a threshold value, wherein the average fitness score is calculated for the second new population of molecules obtained from the last instance of step (c); and
a designated number of molecules in the second new population of molecules obtained from the last instance of step (c) has a fitness score that is less than a threshold value.

9. The method of claim 1, wherein the obtaining the initial population of molecules comprises:
clustering, based on a similarity metric, a corresponding set of molecules in the molecular structure database into one or more clusters of molecules; and
selecting, from each of the one or more clusters of molecules, one or more molecules having a fitness score satisfying one or more initial fitness scores.

10. The method of claim 9, wherein the similarity metric is a Tanimoto index, a cosine coefficient, a Dice metric, an Euclidean metric, a city-block metric, a Hamming index, or a Tversky index, and wherein the similarity metric is based on a property selected from:
2D similarity, 3D similarity, and a vector of physicochemical properties.

11. The method of claim 9, wherein the clustering is performed by applying one or more clustering algorithms selected from: Butina, centroid, CLink, Gower, McQuitty, SLink, Unweighted Pair Group Method with Arithmetic Mean (UPMGA), Ward, and Jarvis-Patrick.

12. The method of claim 2, wherein the one or more fitness scores for a molecule includes a calculated value of one or more of the following molecular properties: solubility, permeability, a selectivity score, an efficiency score, toxicity, and a physiologically based pharmacokinetic (PBPK) score.

13. The method of claim 1, wherein the initial population of molecules is obtained by a method chosen from:
randomly selecting one or more molecules from the molecular structure database;
selecting one or more molecules from the molecular structure database that have one or more physicochemical properties that meet threshold criteria; and
selecting one or more molecules that have a particular scaffold;
or a combination thereof.

14. The method of claim 1, further comprising:
(f) synthesizing at least one of the candidate molecules.

15. The method of claim 2, wherein the second new population of molecules is generated by selecting, as at least a portion of the another N quantity of molecules, a fixed proportion of molecules originating from the first new population of molecules.

16. The method of claim 3, wherein the one or more modifications for each molecule are identified by:
determining a set of applicable modifications for the molecule from a list of available modifications, and
randomly choosing one or more modifications from the set of applicable modifications.

17. A computer-implemented method, comprising:
(a) obtaining, from a molecular structure database, a plurality of initial populations of molecules;
(b) generating a plurality of first new populations of molecules from each of the plurality of respective initial populations of molecules, wherein each of the plurality of first new populations of molecules comprises one or more molecules obtained by modifying one or more molecules in the initial population of molecules on which the first new population is based, wherein the molecules in each of the plurality of first new populations of molecules satisfy one or more fitness scores;
(c) generating a plurality of second new populations of molecules from each of the respective plurality of first new populations of molecules, wherein each of the plurality of second new populations comprise one or more molecules obtained by modifying one or more molecules in the first new population of molecules on which the second new population is based, wherein the molecules in each of the plurality of second new populations of molecules satisfy the one or more fitness scores;
(d) in response to determining that a convergence criterion has not been satisfied, repeating step (c) at least once with each of the first new populations of molecules being one of the second new populations of molecules obtained from the prior instance of step (c); and
(e) in response to determining that the convergence criterion has been satisfied, selecting a subset of molecules that comprises at least one molecule from each of the plurality of second new populations of molecules for synthesis and testing.

18. The method of claim 17, wherein the one or more fitness scores comprise a diversity score.

19. The method of claim 18, wherein the diversity score is based on a structural similarity between a pair of molecules that comprises one molecule selected from each of two populations of molecules, and wherein the diversity score penalizes at least one molecule in a pair of molecules that are structurally similar to one another.

20. The method of claim 18, wherein the diversity score is calculated from a metric selected from the group consisting of: a Tanimoto index, a cosine coefficient, a Dice metric, an Euclidean metric, a city-block metric, a Hamming index, and/or a Tversky index.

21. The method of claim 18, wherein the diversity score is calculated between every pair of molecules that can be formed from molecules in one population and molecules in another population.

22. The method of claim 18, wherein for any pair of molecules whose diversity score does not satisfy a threshold, only one molecule from the pair will satisfy the one or more fitness scores.

23. The method of claim 17,
the generating the plurality of first new population of molecules comprises performing the following for each first new population of molecules:
(i) calculating one or more fitness scores for at least the first modified molecule and for each molecule of the corresponding initial population of molecules; and
(ii) forming the first new population of molecules by selecting, from the corresponding initial population of molecules and at least the first modified molecule, an N quantity of molecules having one or more fitnesss score that satisfy the one or more fitness scores;
and
each instance of the generating the plurality of second new population of molecules comprises performing the following for each second new population of molecules:
(i) calculating one or more fitness scores for at least the second modified molecule and each molecule in the corresponding first new population of molecules; and
(ii) forming the second new population of molecules by selecting, from the corresponding first new population of molecules and at least the second modified molecule, another N quantity of molecules having one or more fitness scores that satisfy the one or more fitness scores.

24. A computer-implemented method, comprising:
(a) obtaining an initial population of molecules wherein each molecule has a molecular structure constructed from a reaction database that contains a set of reactions, wherein each reaction from the set of reactions is associated with two or more sets of reagents, and wherein each molecule in the initial population of molecules is a product of a first reagent from a first set of reagents and a second reagent from a second set of reagents, and optionally a third reagent from a third set of reagents, in accordance with a reaction to which both the first and second reagents and the optional third set of reagents are associated;
(b) generating a first new population of molecules by
modifying at least a first molecule in the initial population of molecules by replacing the first reagent from which the first molecule is formed with another first reagent from the first set of reagents associated with the reaction from which the first molecule is formed, and optionally replacing the second reagent from which the first molecule is formed with another second reagent from the second set of reagents associated with the reaction from which the first molecule is formed, thereby forming a first modified molecule, and
selecting, from the initial population of molecules and at least the first modified molecule, a quantity of molecules that satisfy one or more fitness scores for inclusion in the first new population of molecules;
(c) generating a second new population of molecules by at least
modifying at least a second molecule in the first new population of molecules by replacing the first reagent from which the second molecule is formed with another first reagent from the first set of reagents, and optionally replacing the second reagent from which the second molecule is formed with another second reagent from the second set of reagents thereby forming a second modified molecule, and
selecting, from the first new population of molecules and at least the second modified molecule, another quantity of molecules that satisfy the one or more fitness scores;
(d) in response to determining that a convergence criterion has not been satisfied, repeating step (b) at least once with the second new population of molecules as a new initial population of molecules; and
(e) in response to determining that the convergence criterion has been satisfied, selecting a subset of the second new population of molecules as candidates for synthesis and testing.

25. A computer-implemented method, comprising:
(a) obtaining, from a molecular structure database, an initial population of molecules, each molecule of the initial population of molecules being a product of a first reaction between a first reagent selected from a first set of reagents associated with the first reaction and a second reagent selected from a second reagent selected from a second set of reagents associated with the first reaction, and optionally a third reagent from a third set of reagents associated with the first reaction;
(b) generating a first new population of molecules by
creating a first modified molecule by applying a modification to at least a first molecule in the initial population of molecules, wherein the modification is selected from one or more of:
replacing the first reagent from which the first molecule is formed with another first reagent selected from the first set of reagents;
replacing the second reagent from which the first molecule is formed with another second reagent selected from the second set of reagents; and
replacing a third reagent from which the first molecule is formed with another third reagent selected from the third set of reagents;
and
selecting, from the initial population of molecules and at least the first modified molecule, a quantity of molecules satisfying one or more fitness scores;
(c) generating a second new population of molecules by
creating a second modified molecule by applying a modification to at least a second molecule in the second new population of molecules, wherein the modification is selected from one or more of:
replacing the first reagent from which the first molecule is formed with another first reagent selected from the first set of reagents;
replacing the second reagent from which the first molecule is formed with another second reagent selected from the second set of reagents; and
replacing a third reagent from which the first molecule is formed with another third reagent selected from the third set of reagents;
and
selecting, from the first new population of molecules and at least the second modified molecule, another N quantity of molecules satisfying the one or more fitness scores;
(d) in response to determining that a convergence criterion has not been satisfied, repeating step (c) at least once with the second new population of molecules as a new initial population of molecules; and
(e) in response to determining that the convergence criterion has been satisfied, selecting a subset of the second new population of molecules as candidates for synthesis and testing.

26. The method of claim 25, wherein the generating a first new population of molecules further comprises:
modifying an additional first molecule in the initial population of molecules by:
selecting a third reagent that has a calculated similarity to the first reagent from which the additional first molecule is formed, wherein the third reagent is in an additional first set of reagents associated with an additional reaction, wherein the additional reaction is different to the reaction from which the additional first molecule is formed;
constructing an additional first modified molecule by replacing the first reagent from which the additional first molecule is formed, with the third reagent, and replacing the second reagent from which the additional first molecule is formed with a fourth reagent selected from an additional second set of reagents associated with the additional reaction, wherein the fourth reagent has a calculated similarity to the second reagent;
and
selecting, from the initial population of molecules, the first modified molecule, and the additional first modified molecule, a quantity of molecules satisfying one or more fitness scores.

27. The method of claim 26, wherein the generating a second new population of molecules further comprises:
modifying an additional second molecule in the initial population of molecules by:
selecting a third reagent that has a calculated similarity to the first reagent from which the additional second molecule is formed, wherein the third reagent is in an additional first set of reagents associated with an additional reaction, wherein the additional reaction is different to the reaction from which the additional second molecule is formed;
constructing an additional second modified molecule by replacing the first reagent from which the additional second molecule is formed with the third reagent, and replacing the second reagent from which the additional second molecule is formed, with a fourth reagent selected from a fourth additional second set of reagents associated with the additional reaction, wherein the fourth reagent has a calculated similarity to the second reagent;
and
selecting, from the first new population of molecules, the second modified molecule, and the additional second modified molecule, a quantity of molecules satisfying one or more fitness scores.

28. A system, comprising:
at least one data processor; and
at least one memory storing instructions, which when executed by the at least one data processor, result in operations comprising:
(a) obtaining, from a molecular structure database, an initial population of molecules;
(b) generating a first new population of molecules by modifying at least a first molecule in the initial population of molecules and selecting, from the initial population of molecules and at least the first modified molecule, a quantity of molecules satisfying one or more fitness scores;
(c) generating a second new population of molecules by modifying at least a second molecule in the first new population of molecules and selecting, from the first new population of molecules and at least the second modified molecule, another quantity of molecules satisfying the one or more fitness scores;
(d) in response to determining that a convergence criterion has not been satisfied, repeating step (c) at least once with the second new population of molecules as a first new population of molecules; and
(e) in response to determining that the convergence criterion has been satisfied, selecting a subset of the second new population of molecules as candidates for synthesis and testing.

29. A non-transitory computer readable medium storing instructions, which when executed by at least one data processor, result in operations comprising:
(a) obtaining, from a molecular structure database, an initial population of molecules;
(b) generating a first new population of molecules by modifying at least a first molecule in the initial population of molecules and selecting, from the initial population of molecules and at least the first modified molecule, a quantity of molecules satisfying one or more fitness scores;
(c) generating a second new population of molecules by modifying at least a second molecule in the first new population of molecules and selecting, from the first new population of molecules and at least the second modified molecule, another quantity of molecules satisfying the one or more fitness scores;
(d) in response to determining that a convergence criterion has not been satisfied, repeating step (c) at least once with the second new population of molecules as a first new population of molecules; and
(e) in response to determining that the convergence criterion has been satisfied, selecting a subset of the second new population of molecules as candidates for synthesis and testing.
